(19) United States
(12) Patent Application Publication
(10) Pub. No.: US 2025/0188120 A1
(43) Pub. Date: Jun. 12, 2025

(54) AFFINITY CHROMATOGRAPHIC PRODUCTION OF CLINICAL HUMAN IGG PRODUCTS

(71) Applicant: Takeda Pharmaceutical Company Limited, Osaka (JP)

(72) Inventors: Christian Fiedler, Vienna (AT); Martin Purtscher, Vienna (AT); Lucia Gnauer, Ebersbrunn (AT)

(21) Appl. No.: 18/844,629

(22) PCT Filed: Mar. 6, 2023
(86) PCT No.: PCT/IB23/52096

§ 371 Date: Sep. 6, 2024

Related U.S. Application Data
(60) Provisional application No. 63/317,456, filed on Mar. 7, 2022.

(51) Int. Cl. C07K 1/22; C07K 16/06

(52) U.S. Cl. CPC C07K 1/22; C07K 16/065

(57) ABSTRACT

An affinity chromatographic method of preparing from an IgG-containing starting material an IgG formulation that includes one or more of IgG1, IgG2, IgG3 and IgG4 is provided. A method of the invention includes contacting the IgG-containing starting material with a first chromatographic medium including a first solid support having Protein A bound thereto, forming a Protein A bound fraction of an IgG subclass with Protein A binding affinity and forming a first flow through including an IgG subclass not binding to the Protein A; and contacting the first flow through with a first affinity ligand chromatographic medium including a second solid support having a first affinity ligand with IgG binding affinity bound thereto, forming a first affinity ligand-bound IgG subclass and a second flow through including an IgG subclass not bound to the first affinity ligand-bound IgG subclass.

### BACKGROUND OF THE INVENTION

Immunoglobulin products from human plasma were first used in 1952 to treat immune deficiency. Initially, intramuscular or subcutaneous administration of IgG were the methods of choice. For injecting larger amounts of IgG necessary for effective treatment of various diseases, however, the intravenous administrable products with lower concentrated IgG (50 mg/mL) were developed. Usually intravenous immunoglobulin (IVIG), contains the pooled immunoglobulin G (IgG) immunoglobulins from the plasma of more than a thousand blood donors. Typically containing more than 95% unmodified IgG, which has intact Fc-dependent effector functions, and only trace amounts of immunoglobulin A (IgA) or immunoglobulin M (IgM), IVIGs are sterile, purified IgG products primarily used in treating three main categories of medical conditions: 1. immune deficiencies such as X-linked agammaglobulinemia, hypogammaglobulinemia (primary immune deficiencies), and acquired compromised immunity conditions (secondary immune deficiencies), featuring low antibody levels; 2. inflammatory and autoimmune diseases; and 3. acute infections.

A number of IVIG commercial suppliers provide a variety of IVIG products. Compared to the older lyophilized IVIG products containing only 50 mg/mL protein in the solution after re-dissolving, the latest developments are ready-for-use sterile, liquid preparations of highly purified and concentrated human IgG antibodies in various volumes and concentrations. Since IgG products such as IVIGs are manufactured from pooled human plasma, pathogen contamination (especially viruses known to cause various diseases in human) from donor blood is a serious concern in the production process. Another important consideration in IgG products is their stability during storage, especially as ready-for-use preparations. Compared to IVIG, subcutaneously administrable immunoglobulin preparations have the advantages of home-care treatment possibility and less side effects.

In the fourth installment of a series of seminal papers published on the preparation and properties of serum and plasma proteins, Cohn et al. (J. Am. Chem. Soc., 1946, 68 (3): 459-475) first described methods for the alcohol fractionation of plasma proteins (method 6), which allows for the isolation of a fraction enriched in IgG from human plasma. Several years later, Oncley et al. (J. Am. Chem. Soc., 1949, 71 (2): 541-550) expanded upon the Cohn methods by publishing a method (method 9) that resulted in the isolation of a purer IgG preparation.

These methods, while laying the foundation for an entire industry of plasma derived blood factors, were unable to provide IgG preparations having sufficiently high concentrations for the treatment of several immune-related diseases, including Kawasaki syndrome, immune thrombocytopenia purpura, and primary immune deficiencies. As such, additional methodologies employing various techniques, such as ion exchange chromatography, were developed to provide higher purity and higher concentration IgG formulations. Hoppe et al. (Munch Med Wochenschr 1967 (34): 1749-1752) and Falksveden (Swedish Patent No. 348942) and Falksveden and Lundblad (Methods of Plasma Protein Fractionation 1980) were among the first to employ ion exchange chromatography for this purpose.

Various modern methods employ a precipitation step, such as caprylate precipitation (Lebing et al., Vox Sang 2003 (84): 193-201) and Cohn Fraction (I+)II+III ethanol precipitation (Tanaka et al., Braz J Med Biol Res 2000 (33) 37-30) coupled to column chromatography. Most recently, Teschner et al. (Vox Sang, 2007 (92): 42-55) have described a method for production of a 10% IVIG product in which cryo-precipitate is first removed from pooled plasma and then a modified Cohn-Oncley cold ethanol fractionation is performed, followed by S/D treatment of the intermediate, ion exchange chromatography, nanofiltration, and optionally ultrafiltration/diafiltration.

Despite the purity, safety, and yield afforded by these IgG isolation methods, the yield of IgG recovered from plasma can still be improved. For example, Teschner et al. report that their method results in an increased IgG yield of 65% (Teschner et al., supra). Although an improvement over methods previously employed, this amount of IgG recovery still represents a loss of at least about a third of the IgG present in the pooled plasma fraction during the isolation process.

Due to the limited supply of plasma available for the manufacture of plasma-derived products, the isolation of several blood proteins from a common starting plasma pool can be achieved by integrating the purifications into a single framework. For example, IgG is commonly enriched through the formation of a Cohn Fraction II+III precipitate or Kistler-Nitschmann precipitate A, the corresponding supernatants of which are then used for the manufacture of alpha-1-antitrypsin and albumin. Similarly, several methods have been described for the manufacture of other plasma proteins such as Factor H from by-products formed during the manufacture of IgG immunoglobulins, including WO 2008/113589 and WO 2011/011753.

As such, a need exists for improved and more efficient methods for manufacturing of therapeutic IgG products.

In addition, methods yielding highly concentrated IgG preparations suitable for subcutaneous and/or intramuscular administration are still needed.

Most protein purification methods involve some form of chromatography, whereby molecules in solution (mobile phase) are separated based on differences in chemical or physical interaction with a solid material (stationary phase). Gel filtration (also called size-exclusion chromatography or SEC) uses a porous resin material to separate molecules based on size (i.e., physical exclusion). In ion exchange chromatography, molecules are separated according to the strength of their overall ionic interaction with a solid phase material (i.e., nonspecific interactions). Affinity chromatographic methods are widely used in protein purification, however, this method has not been deployed for the large-scale, industrial-scale purification of plasma proteins, such as IgG.

Affinity chromatography (also called affinity purification) relies on specific binding interactions between molecules. A particular ligand is chemically immobilized or “coupled” to a solid support so that when a complex mixture is passed over the column, those molecules having specific binding affinity to the ligand become bound. After other sample components are washed away, the bound molecule is stripped from the support, resulting in its purification from the original sample.

A range of affinity ligands useful for producing and purifying proteins is known in the art. Protein A and protein G are the most commonly used capture proteins in the purification of human antibodies. However, protein A and protein G have several drawbacks, including their high cost, low stability, and the possibility of contaminating the product through hydrolysis and release of peptide fragments. Generally, Protein A and/or Protein G derived affinity resins do not bind all IgG subclasses of the human IgG fraction efficiently. Thus, there is a need for an efficient, preferably compact method allowing binding of a broader range of human IgG subclasses by combining a Protein A or Protein G derived resin with one or more IgG binding resins based on a complementary binding principle.

The present invention answers these and other needs, providing an efficient, effective chromatographic method for producing IgG, which, in one embodiment, circumvents the exacting, time-consuming ethanol fractionation processes currently standard in the plasma products field.

### SUMMARY OF THE INVENTION

There remains a need for more efficient, rapid and high yielding processes to purify IgG from a feedstock. As individual IgG isoforms are characterized by different affinities for their conjugate epitopes, a method that allowed the facile capture and/or purification of one or more isotype would allow the affinity of a specific isotype to be enhanced and exploited in a therapeutic IgG preparation. The present invention provides these and other advantages by providing a new method of performing IgG-specific affinity chromatography on a feedstock containing one or more IgG isoforms.

Quite surprisingly, the inventors have discovered that by running two or more affinity capture chromatographic steps on a plasma product starting material, e.g., cryo-supernatant, highly pure (e.g., >80, 81, 82, 83, 84, 85, 86, 87, 88, 89, 90, 91, 92, 93, 94, 95, 96, 97, 98, 99%,) IgG products with IgG subtype profiles virtually identical to plasma IgG subtype distribution can be produced in a rapid, efficient and high yielding manner. The present invention thus provides a method for producing IgG formulations with IgG subtype profiles, impurity profiles, and a combination thereof, which are essentially identical to those of current approved, marketed IgG formulations, and without resorting to classical plasma fractionation technologies (e.g., Cohn fractionation). This unexpected discovery represents a significant advance in the field of isolating IgG fractions from bulk plasma.

In an exemplary embodiment, there is provided an aqueous IgG solution comprising at least about 99.5% IgG and less than about 0.5% impurities whereby the aqueous IgG solution comprises Factor XI as an impurity at an activity of less than about 0.0025 E/mg of total IgG, preferably less than about 0.0020 E/mg of total IgG, more preferably less than about 0.0015 E/mg of total IgG, and most preferably less than about 0.0012 E/mg of total IgG. In a further exemplary embodiment, there is provided an aqueous IgG solution comprising at least about 99.5% IgG and less than about 0.5% impurities whereby the aqueous IgG solution comprises Complement C3 as an impurity at a level of less than about 3.5 μg/mg of total IgG, preferably less than about 3.0 μg/mg of total IgG, more preferably less than about 2.5 μg/mg of total IgG, and most preferably less than about 2.2 μg/mg of total IgG. In an exemplary embodiment, there is provided an aqueous IgG solution comprising at least about 99.5% IgG and less than about 0.5% impurities whereby the aqueous IgG solution comprises as impurity Complement C3 and Factor XI, whereby Complement C3 is present at a level of less than about 3.5 μg/mg of total IgG, preferably less than about 3.0 μg/mg of total IgG, more preferably less than about 2.5 μg/mg of total IgG, and most preferably less than about 2.2 μg/mg of total IgG and whereby Factor XI is present at an activity of less than about 0.0025 E/mg of total IgG, preferably less than about 0.0020 E/mg of total IgG, more preferably less than about 0.0015 E/mg of total IgG, and most preferably less than about 0.0012 E/mg of total IgG. In an exemplary embodiment, there is provided an aqueous IgG solution comprising at least about 99.5% IgG and less than about 0.5% impurities whereby the aqueous IgG solution comprises as an impurity Complement C3 and Factor XI, whereby Complement C3 is present at a level of less than about 2.5 μg/mg of total IgG and whereby Factor XI is present at an activity of less than about 0.0015 E/mg of total IgG.

One embodiment of the present invention is directed to an affinity chromatographic method of preparing from an IgG-containing starting material an IgG formulation comprising one or more of IgG1, IgG2, IgG3 and IgG4 in any combination. In an exemplary embodiment, the method provides an IgG formulation with a pre-determined pattern/ratio of IgG subtypes, pre-determined impurity pattern and a combination thereof. In an exemplary embodiment, the pattern/ratio is essentially identical to the pattern/ratio of plasma IgG subtype distribution.

Both surprising and unexpected was that the second column, i.e., that following the Protein A column, bound to a significant fraction of the IgG subtypes not bound to the Protein A column. In an exemplary embodiment, the medium on the second column is an antibody. Exemplary antibodies include those able to bind to a common region of one or more IgG subclasses. The antibody can be a full-length monoclonal antibody, or an antibody fragment (e.g., Fab, Fab2, single chain, camelid antibody fragment, or a peptide). In an exemplary embodiment, this column is a CaptureSelect™ CH1-XL Affinity Matrix (ThermoFisher), or a column with a similar IgG binding selectivity.

Another unexpected result of the present invention was the discovery by the inventors that an on-column solvent/detergent viral inactivation (SD_VI) step can be performed on the protein material bound to the column without deleterious effects on the fidelity of the separation aspect of the process. This discovery further enhances the efficiency and compactness of the inventive process, particularly relative to art-standard fractionation procedures, by incorporating the SD_VI step directly into an established unit operation as opposed to it being conducted as a stand-alone step on material separated from the chromatographic medium. In fact, further unexpected was the enhanced purity of IgG formulations in which the on-column SD_VI step was incorporated into the purification as this step was not contemplated to have an effect beyond viral inactivation. The SD_VI step depletes or removes impurities beyond those virally derived.

In exemplary embodiments, the SD_VI step depletes or removes at least one or more of Albumin, Apolipoprotein A-I, Complement C3, Haptoglobin, Fibrinogen, Complement C4-B, Complement C1s subcomponent, Complement C1r subcomponent, Apolipoprotein B-100, Alpha-2-macroglobulin. In an exemplary embodiment, these impurities are found in the wash from the SD_VI step.

In various embodiments, the SD_VI step is performed following an IQEQ step in which the target therapeutic protein (e.g., IgG) is captured on the chromatographic medium and a flow through is produced, and prior to eluting the target therapeutic protein from the chromatographic medium.

Exemplary methods of the invention can be carried out sequentially, or as a single unit operation using an array of two or more different IgG binding affinity resins (tandem array) or on a skid that allows fully automated continuous chromatography. The principle is not limited to IgGs, but can be applied to purify other proteins, e.g., plasma proteins, e.g., albumin, A1PI, FVIII, etc.

Exemplary embodiments of the invention described herein offer the advantages that the purification of an immunoglobulin, e.g., a human IgG fraction out of a feedstock, e.g., plasma and/or plasma fractions (e.g., cryoprecipitate supernatant) with a broad range of IgG subclass composition is superior over methods using single Protein A or Protein G derived, or another IgG binding affinity purification. The high purity of the obtained IgG subclass composition has the benefit of a significant reduction of unit operations to finalize the purified IgG product to an administrable drug.

An exemplary method includes (a) contacting a IgG-containing starting material with a first chromatographic medium comprising a first solid support having Protein A bound thereto, forming a Protein A bound fraction of an IgG subclass with Protein A binding affinity and forming a first flow through comprising an IgG subclass not bound to the Protein A; (b) contacting the first flow through with a first affinity ligand chromatographic medium comprising a second solid support having a first affinity ligand with IgG binding affinity bound thereto, forming a first affinity ligand-bound IgG subclass and a second flow through comprising an IgG subclass not bound to the first affinity ligand-bound IgG subclass; (c) eluting the Protein A bound fraction from the first chromatographic medium, forming a first eluate and contacting the first eluate with the first affinity ligand chromatographic medium, forming a second affinity ligand-bound IgG subclass, and a second flow through; and (d) eluting a member selected from the first affinity ligand-bound IgG subclass, the second affinity ligand-bound IgG subclass, and a combination thereof from the first affinity ligand column, forming a second eluate.

An exemplary affinity ligand chromatographic medium includes a VHH antibody fragment.

In an exemplary embodiment, the method further includes, (e) combining the first flow through and the second eluate on the first affinity ligand chromatographic medium.

In an exemplary embodiment, the method includes (f) contacting the second eluate of (d) with a second affinity ligand chromatographic medium comprising a third solid support having a second affinity ligand with IgG binding affinity bound thereto, forming a third affinity ligand-bound IgG subclass and a second flow through comprising a fourth IgG subclass not bound to the second affinity ligand chromatographic medium; and (g) eluting a member selected from the second affinity ligand bound IgG subclass, the fourth IgG subclass and a combination thereof from the second affinity ligand chromatographic medium, forming a third eluate.

In an exemplary embodiment, the method includes (h) prior to (g), combining the first flow through and the second eluate on the second affinity ligand chromatographic medium.

In various embodiments, the method includes (i) prior to (b), contacting the first chromatographic medium with a buffer/detergent mixture that does not elute the Protein A bound fraction of an IgG subclass with Protein A binding affinity to from the first chromatographic medium.

In some embodiments, the method includes (j) prior to (g), contacting the second affinity ligand chromatographic medium with a buffer/detergent mixture that does not elute the second affinity ligand-bound IgG subclass from the second affinity ligand chromatographic medium.

An exemplary aspect of the present invention is directed to an affinity chromatographic method of preparing an IgG formulation enriched in a first IgG subclass with low protein A binding affinity from a plasma-derived IgG solution depleted in a Protein A binding IgG subclass. The method includes (a) contacting the IgG solution depleted in a Protein A binding IgG subclass with a first affinity ligand chromatographic medium comprising a first solid support having a first affinity ligand with IgG binding affinity bound thereto, forming a first bound fraction of the first IgG subclass with low protein A binding affinity and a first flow through; and (b) eluting the first bound fraction of the first IgG subclass with low protein A binding affinity, forming a first eluent comprising the IgG formulation enriched in the first IgG subclass with low protein A binding.

In an exemplary embodiment, the method further includes (c) prior to (b), contacting the first bound fraction of the first IgG subclass with low protein A binding affinity with a buffer/detergent solution, and subsequently removing the detergent solution from the first affinity ligand chromatographic medium.

In an exemplary embodiment, the method further includes (d) following (b), contacting the first eluent with a second chromatographic medium comprising a solid support with an affinity ligand having affinity for a second IgG subclass with low protein A binding affinity bound thereto, forming a first bound fraction of a second IgG subclass with low protein A binding affinity; and (e) eluting from the second chromatographic medium the first bound fraction of the second IgG subclass with low protein A binding affinity, forming a second eluate.

In some embodiments, the method further includes (f) prior to (e), contacting the first bound fraction of the second IgG subclass with low protein A binding affinity with a buffer/detergent solution, and subsequently removing the detergent solution from the first chromatographic medium.

In various embodiments, the method further includes (g) prior to (a), contacting a plasma-derived IgG solution with a Protein A chromatographic medium comprising Protein A bound to a solid support, forming a Protein A-bound fraction of an IgG subclass with high affinity for Protein A, and the IgG formulation enriched in a first IgG subclass with low protein A binding affinity.

In an exemplary embodiment, the method further includes (h) eluting the Protein A-bound fraction of an IgG subclass with high affinity for Protein A from the Protein A chromatographic medium, forming a third eluate.

An exemplary method includes a step of combining two or more of the first eluate, the second eluate and the third eluate.

Other aspects and advantages of the invention will be apparent from the following detailed description, figures and the appended claims.

### BRIEF DESCRIPTION OF THE DRAWINGS

FIG. 1 is a UV profile of Protein A binding IgG Subclasses Tandem Column I (Protein A and CH1-XL).

FIG. 2 is a UV profile of non-Protein A binding subclasses on IgG Capture Select resins—overlay of flow through (FT).

FIG. 3 is a UV profile of non-Protein A binding subclasses on IgG Capture Select resins overlay of elution peaks.

FIG. 4 is a chromatogram of IGC02.

FIG. 5 is a chromatogram IGC01S.

FIG. 6 is a chromatogram IGC04.

FIG. 7 is a SDS page silver stain profile of IGC04 Silver Stain. 1. Load, Cryo supernatant, diluted 1:560. 2. Flow through, diluted 1:500. 3. WSD, On column SD_VI, diluted 1:10. 4. Wash 2, diluted 1:5. 5. Wash 3, diluted 1:5. 6. Eluate, diluted 1:100. 7. Eluate, diluted 1:50. 8. Strip, diluted 1:10. NuPAGE 4-12% Bis-Tris Midi Gel 1.0 mm. 20 well Cat.Nr. WG1402BX10, Non-reducing SDS Running Buffer LDS-SB: NP0007, 50 μl Sample added to 20 μl Iodacetamide incubated 30 min at +18 to 26° C., +50 μl Buffer LDS-SB: NP0007 incubated 10 min at 70° C. Lanes 8 and 9 demonstrate a surprisingly high level of purity for the eluate.

FIG. 8 is a chromatogram of IGC06. Conductivity (Red): UV280 (Blue) UV254 (violet) pH: Green.

FIG. 9 is an SDS page silver stain profile of IGC06 Silver Stain. 1. Mol weight marker. 2. Blank 3.Load, Cryo supernatant, diluted 1:560. 4. Flow through, diluted 1:500. 5.WSD, On column SD_VI, diluted 1:10. 6. Wash 2, diluted 1:5. 7. Wash 3, diluted 1:5. 8. Eluate, diluted 1:100 IgG fraction. 9. Eluate, diluted 1:50 IgG fraction. 10. Strip, diluted 1:10. NuPAGE 4-12% Bis-Tris Midi Gel 1.0 mm. 20 well Cat. Nr. WG1402BX10. Non reducing SDS Running Buffer LDS-SB: NP0007. 50 μl Sample added to 20 μl Iodacetamide incubated 30 min at +18 to 26° C. +50 μl Buffer LDS-SB: NP0007 incubated 10 min at 70° C.

FIG. 10 is a model flow diagram for loading the test material (10% polyclonal immunoglobulin) onto a Protein A column.

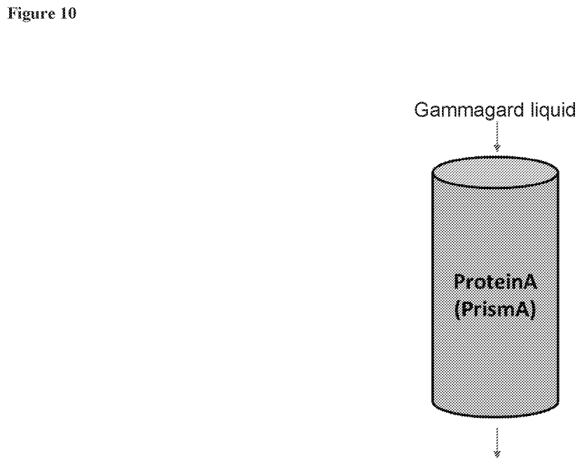

FIG. 11 is a model flow diagram showing the eluate from FIG. 10 aliquoted into individual columns, each containing a separate affinity chromatographic medium. The eluate from each of these columns is collected separately.

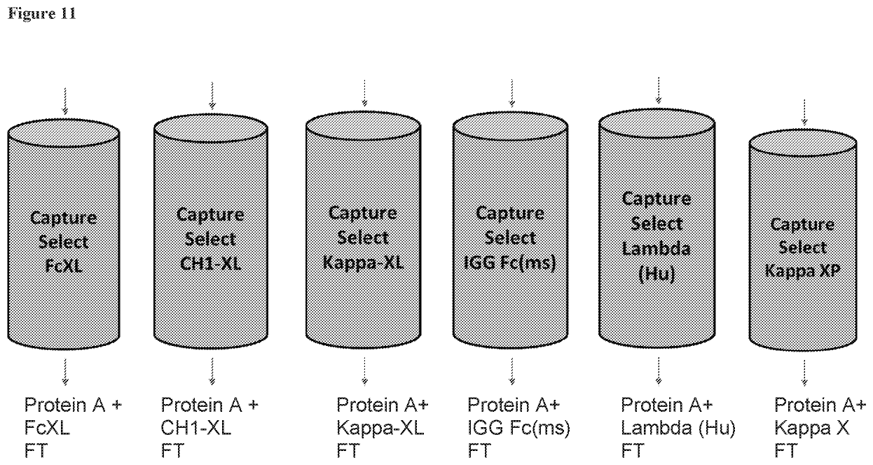

FIG. 12 is a model flow diagram showing a test material (Cryo-supernatant) loaded onto a Protein A column. The eluate from this column is loaded into a column of an affinity chromatography medium (e.g., Capture Select CH1-XL).

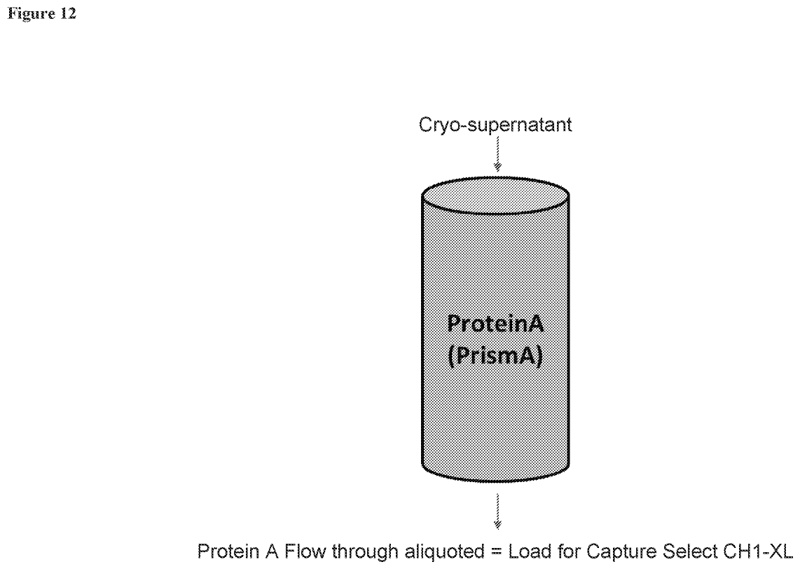

FIG. 13 is a model flow diagram showing eluate from Protein A chromatography of a cryo-supernatant loaded onto an exemplary affinity ligand chromatography medium (CH1-XL).

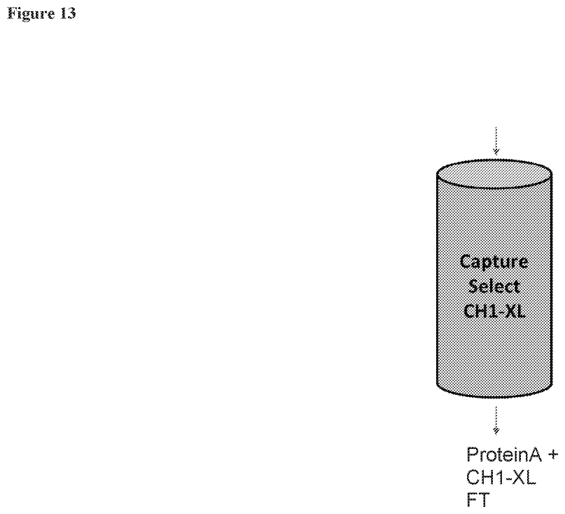

FIG. 14 is a model flow diagram showing a cryo-supernatant loaded onto a tandem array of Protein A and an affinity chromatography medium. A wash step is performed on the array after loading the cryo-supernatant. The eluate contains a broad range of IgG subclasses.

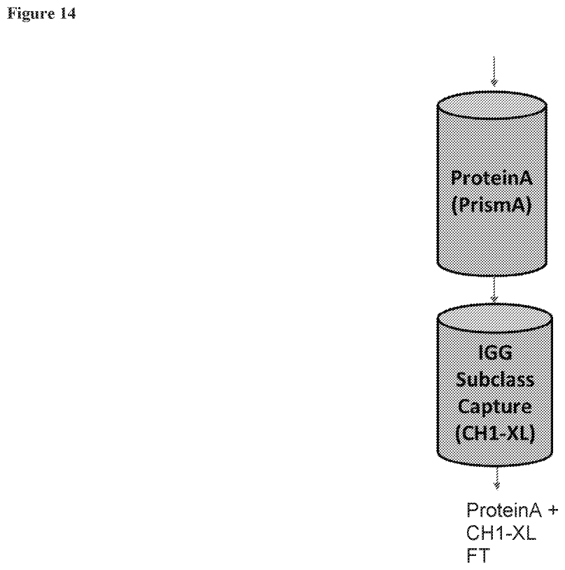

FIG. 15 is a model flow diagram for a three-column array (TRIDEM) of Protein A, Capture Select CH1-XL, and Capture Select IgG3. Cryo-precipitate is loaded onto the array, and a wash step was performed. Eluate contains a broad range of IgG subclasses.

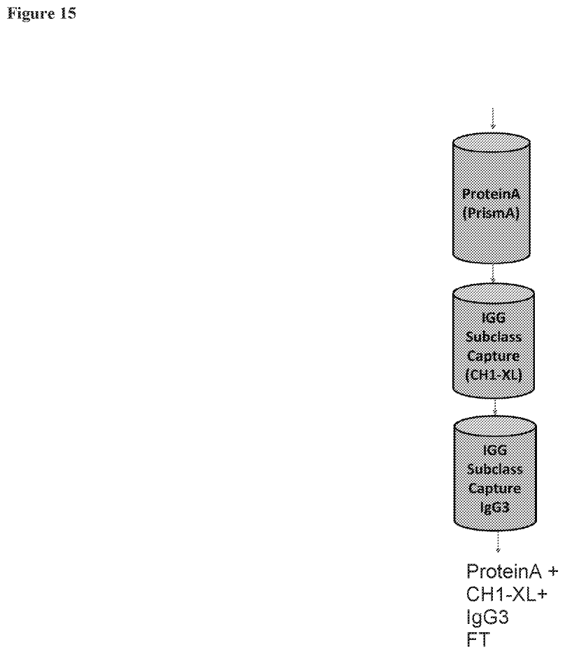

FIG. 16 is an overview of an exemplary tandem and tridem chromatographic array.

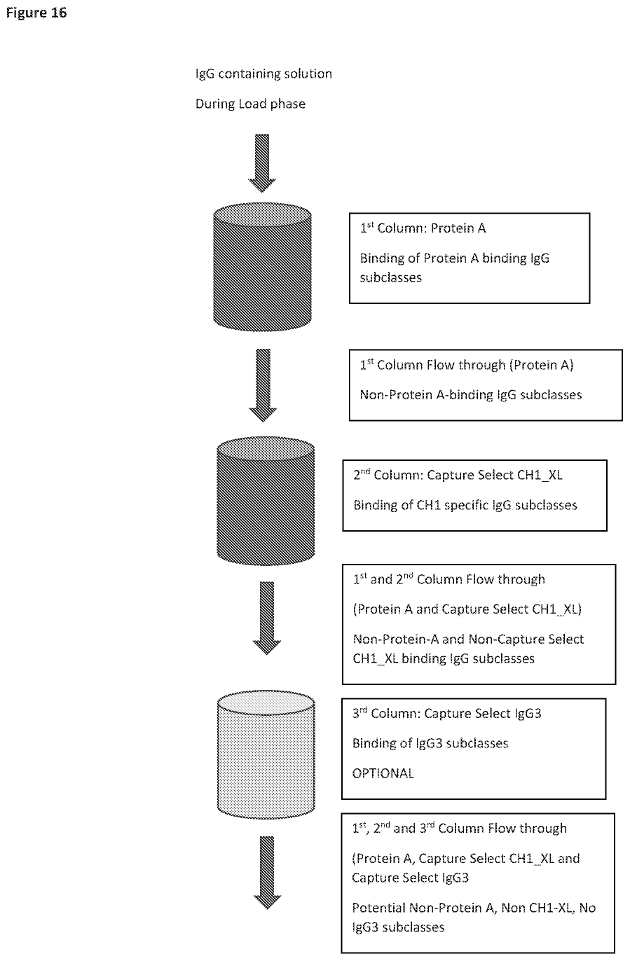

### DETAILED DESCRIPTION OF THE INVENTION

### A. Introduction

Provided herein are exemplary embodiments of purifying one or more IgG isotype from a feedstock using a process incorporating at least one affinity ligand-based affinity chromatographic method. An exemplary IgG preparation of the invention is prepared from an IgG-containing starting material, e.g., cryoprecipitate supernatant and provides an IgG formulation comprising a purified immunoglobulin.

An exemplary method includes (a) contacting a IgG-containing starting material with a first chromatographic medium comprising a first solid support having Protein A bound thereto, forming a Protein A bound fraction of an IgG subclass with Protein A binding affinity and forming a first flow through comprising an IgG subclass not binding to the Protein A; (b) contacting the first flow through with a first affinity ligand chromatographic medium comprising a second solid support having a first affinity ligand with binding affinity for a selected immunoglobulin bound thereto, forming a first affinity ligand-bound IgG subclass and a second flow through comprising an IgG subclass not bound to the first affinity ligand; (c) eluting the Protein A bound fraction from the first chromatographic medium, forming a first eluate and contacting the first eluate with the first affinity ligand chromatographic medium, forming a second affinity ligand-bound IgG subclass, and a second flow through; and (d) eluting a member selected from the first affinity ligand-bound IgG subclass, the second affinity ligand-bound IgG subclass, and a combination thereof from the first affinity ligand column, forming a second eluate.

Protein A based chromatographic media do not bind IgG of all subclasses with comparable efficiency. In exemplary embodiments, the present invention provides methods and systems of isolating and/or purifying IgG fractions and IgG subtypes from a plasma starting material using affinity ligand-based affinity resins. Exemplary resins of use in the invention include those rather recently developed for the purification of antibody fusion proteins. Those resins bind IgG at different sites (modes) and, therefore, are of use to compliment and augment the use of Protein A resins, or to overcome the shortcomings of Protein A based resins.

In addition, the affinity-chromatography based approach of the present invention can be formatted in a continuous or semi-continuous mode enabling a high throughput.

An exemplary embodiment includes dedicated washing steps and an on-column solvent/detergent, viral inactivation step (“On column” SD_VI treatment), thereby reducing the total number of unit operations compared to traditional IgG fractionation and purification. The on-column SD_VI step can be performed on any column or more than one column within the array.

An exemplary process of the invention includes at least two virus inactivation/depletion steps (e.g., dedicated SD_VI, acidic hold step, nanofiltration 35 nm).

In various embodiments, the invention also provides an IgG preparation that is produced using a method and/or system of the invention.

### B. Definitions

While the terms used herein are believed to be well understood by one of ordinary skill in the art, definitions are set forth herein to facilitate explanation of the subject matter disclosed herein.

Unless defined otherwise, all technical and scientific terms used herein have the same meaning as commonly understood by one of ordinary skill in the art to which the subject matter disclosed herein belongs. Although any methods, devices, and materials similar or equivalent to those described herein can be used in the practice or testing of the presently disclosed subject matter, representative methods, devices, and materials are described herein.

The terms “a,” “an,” and “the” refer to “one or more” when used in this application, including the claims. The use of the word “a” or “an” when used in conjunction with the term “comprising” in the claims and/or the specification may mean “one,” but it is also consistent with the meaning of “one or more,” “at least one,” and “one or more than one.”

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and devices of the present disclosure, including components thereof, can comprise, consist of, or consist essentially of the essential elements and limitations of the embodiments described herein, as well as any additional or optional components or limitations described herein or otherwise useful.

Unless otherwise indicated, all numbers expressing physical dimensions, quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

In the interest of clarity, not all of the routine features of the implementations described herein are shown and described. It will be appreciated that, in the development of any such actual implementation, numerous implementation-specific decisions are made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Many modifications and variations of the exemplary embodiments set forth in this disclosure can be made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods. In addition, Harlow & Lane, A Laboratory Manual Cold Spring Harbor, N.Y., is referred to for standard Immunological Techniques.

An “IgG-containing starting material”, as this term is used herein, includes plasma, a cryopoor plasma, a fraction for a plasma fractionation process, e.g., a Cohn fraction, and a recombinant IgG preparation, as well as other sources of feedstocks for the disclosed process which include one or more IgG. In some embodiments, this term refers to human IgG-containing starting materials, but the invention is not limited to the use of human-derived IgG-containing starting materials.

“Low Protein A binding affinity”, as this term is used herein, refers to an Ig species that either does not bind to or is readily washed off of a chromatographic medium comprising immobilized Protein A. An exemplary species with low Protein A binding affinity is one in which bound fraction is essentially completely eluted from a Protein A column at a pH higher than 5, with an eluent having conductivity of greater than about 5 mS/cm, or a combination thereof.

As used herein, an “antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively.

An exemplary immunoglobulin (antibody) structural unit is composed of two pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

The term “antibody” and “immunoglobulin” are used interchangeably to refer, in some embodiments, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally occurring IgG antibodies, the heavy chain constant region is comprised of a hinge and three domains, CHL CH2 and CH3. In some antibodies, e.g., naturally occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. A heavy chain may have the C-terminal lysine or not. The term “antibody” can include a bispecific antibody or a multi-specific antibody. An exemplary affinity ligand is an antibody or a fragment thereof.

An exemplary antibody (immunoglobulin) includes a human IgA, IgD, IgG, IgE or IgM. The antibody can be an IgG1, IgG2, IgG3 and IgG4 antibody. As used herein an “IgG” has, in some embodiments, the structure of a naturally occurring IgG antibody, i.e., it has the same number of heavy and light chains and disulfide bonds as a naturally occurring IgG antibody of the same subclass. For example, an IgG antibody may consist of two heavy chains (HCs) and two light chains (LCs), wherein the two HCs and LCs are linked by the same number and location of disulfide bridges that occur in naturally occurring Ig antibodies, respectively (unless the antibody has been mutated to modify the disulfide bridges).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally occurring and non-naturally occurring antibodies; monoclonal and polyclonal antibodies; chimeric and humanized antibodies; human and nonhuman antibodies and wholly synthetic antibodies.

Camelid antibodies are sometimes used in affinity purification procedures. A significant proportion of camelid antibodies are single-domain antibodies, which interact with their antigen via a single heavy-chain binding domain devoid of light chain. This domain is also referred to as “VHH” or “VHH antibody” or “VHH domain”. Recombinant VHH antibodies present a minimal-sized intact antigen-binding domain. The absence of the VL domain allows the VHH antibodies to attain a higher structural flexibility than that of VH domains associated with VL domains. Furthermore, the complementarity determining regions (CDRs) of VHHs, and especially CDR3, are statistically longer than those of conventional VH-VL antibodies (Muyldermans S., J. Biotechnol., 2001, 74, 277-302).

An exemplary affinity ligand is a VHH ligand. By “VHH ligand” is meant a single-domain heavy chain antibody, or antibody fragment, derived from camelids. In general, VHH ligands have a heavy chain derived from an immunoglobulin naturally devoid of light chains that is joined together to form a multivalent single polypeptide which retains the antigen binding affinity of the parent whole immunoglobulin, but which is much smaller in size and therefore less immunogenic. VHH ligands are described in detail, for example, in Frenken et al., J. Biotechnol. 78:11-21 (2000), van der Linden et al., Biochem Biophys. Acta. 1431:37-46 (1999), Spinelli et al., Biochemistry 39:1217-1222 (2000), U.S. Patent Application Publication No. 20030078402, 2004/0142432, and U.S. Pat. Nos. 6,399,763 and 6,670,453.

Exemplary VHH antibodies (and fragments) of use as chromatographic affinity ligands in the methods of the invention include those having affinity for at least one isotype of IgG. Exemplary VHH antibodies of use in the methods of the invention are selective for at least a first isotype over a second isotype of IgG.

The term “antigen-binding portion” of an antibody, fragment thereof (or affinity ligand, as used herein), refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

As used herein, the term “ultrafiltration (UF)” encompasses a variety of membrane filtration methods in which hydrostatic pressure forces a liquid against a semi-permeable membrane. Suspended solids and solutes of high molecular weight are retained, while water and low molecular weight solutes pass through the membrane. This separation process is often used for purifying and concentrating macromolecular (10³-10⁶ Da) solutions, especially protein solutions. A number of ultrafiltration membranes are available depending on the size of the molecules they retain. Ultrafiltration is typically characterized by a membrane pore size between 1 and 1000 kDa and operating pressures between 0.01 and 10 bar and is particularly useful for separating colloids like proteins from small molecules like sugars and salts.

As used herein, the term “diafiltration” is performed with the same membranes as ultrafiltration and is a tangential flow filtration. During diafiltration, buffer is introduced into the recycle tank while filtrate is removed from the unit operation. In processes where the product is in the retentate (for example IgG), diafiltration washes components out of the product pool into the filtrate, thereby exchanging buffers and reducing the concentration of undesirable species.

The term “chromatography” refers to a technique separating a protein of interest (e.g., an antibody) from other molecules (e.g., contaminants) present in a mixture. Usually, the protein of interest is separated from other molecules (e.g., contaminants) as a result of differences in rates at which the individual molecules of the mixture migrate through a stationary medium under the influence of a moving phase, or in bind and elute processes. The term “matrix” or “chromatography matrix” are used interchangeably herein and refer to any kind of sorbent, resin or solid phase which in a separation process separates a protein of interest (e.g., an Fc region containing protein such as an immunoglobulin) from other molecules present in a mixture. Non-limiting examples include particulate, monolithic or fibrous resins as well as membranes that can be put in columns or cartridges. Examples of materials for forming the matrix include polysaccharides (such as agarose and cellulose); and other mechanically stable matrices such as silica (e.g., controlled pore glass), poly(styrenedivinyl)benzene, polyacrylamide, ceramic particles and derivatives of any of the above. Examples for typical matrix types suitable for the method of the present disclosure are cation exchange resins, affinity resins, anion exchange resins or mixed mode resins. A “ligand” is a functional group that is attached to the chromatography matrix and that determines the binding properties of the matrix. Examples of “ligands” include, but are not limited to, ion exchange groups, hydrophobic interaction groups, hydrophilic interaction groups, thiophilic interactions groups, metal affinity groups, affinity ligands, bioaffinity groups, and mixed mode groups (combinations of the aforementioned). Some preferred ligands that can be used herein include, but are not limited to, strong cation exchange groups, such as sulphopropyl, sulfonic acid; strong anion exchange groups, such as trimethylammonium chloride; weak cation exchange groups, such as carboxylic acid; weak anion exchange groups, such as N5N diethylamino or DEAE; hydrophobic interaction groups, such as phenyl, butyl, propyl, hexyl; and affinity ligands, such as Protein A, Protein G, and Protein L.

The term “affinity chromatography” refers to a protein separation technique in which a protein of interest (e.g., an Fc region containing protein of interest or antibody) is specifically bound to a ligand which is specific for the protein of interest. Such a ligand is generally referred to as an affinity or a biospecific ligand. In some embodiments, the affinity ligand (e.g., Protein A or a functional variant thereof) is covalently attached to a chromatography matrix material and is accessible to the protein of interest in solution as the solution contacts the chromatography matrix. The protein of interest generally retains its specific binding affinity for the affinity ligand during the chromatographic steps, while other solutes and/or proteins in the mixture do not bind appreciably or specifically to the ligand. Binding of the protein of interest to the immobilized ligand allows contaminating proteins or protein impurities to be passed through the chromatography matrix while the protein of interest remains specifically bound to the immobilized ligand on the solid phase material. The specifically bound protein of interest is then removed in active form from the immobilized ligand under suitable conditions (e.g., low pH, high pH, high salt, competing ligand etc.), and passed through the chromatographic column with the elution buffer, free of the contaminating proteins or protein impurities that were earlier allowed to pass through the column. Any component can be used as a ligand for purifying its respective specific binding protein, e.g., antibody. However, in various methods according to the present disclosure, Protein A, G, A/G and/or L is used as a ligand for an Fc region containing a target protein. The conditions for elution from the affinity ligand (e.g., Protein A) of the target protein (e.g., an Fc region containing protein) can be readily determined by one of ordinary skill in the art. In some embodiments, Protein G or Protein L or a functional variant thereof may be used as a biospecific ligand. In some embodiments, a biospecific ligand such as Protein A is used at a pH range of 5-9 for binding to an Fc region containing protein, washing or re-equilibrating the biospecific ligand/target protein conjugate, followed by elution with a buffer having pH about or below 4 which contains at least one salt.

The term “affinity ligand” refers to a ligand capable of capturing one or more IgG species. An exemplary affinity ligand is selective from one IgG species in a mixture of species and use of a column, filter or other medium comprising the affinity ligand provides a method by which one or more IgG species can be purified from a mixture containing the one or more IgG species. An exemplary affinity ligand is a VHH antibody or a fragment thereof.

The terms “purifying,” “separating,” or “isolating,” as used interchangeably herein, refer to increasing the degree of purity of a protein of interest from a composition or sample comprising the protein of interest and one or more impurities. Typically, the degree of purity of the protein of interest is increased by removing (completely or partially) at least one impurity from the composition. “Purifying” and its equivalents refer to one or more step performed to isolate a therapeutic protein from one or more other impurities (e.g., bulk impurities) or components present in a fluid containing a therapeutic protein (e.g., plasma, Cohn fraction, liquid culture medium proteins or one or more other components (e.g., DNA, RNA, other proteins, endotoxins, viruses, etc.) present in or secreted from a mammalian cell). For example, purifying can be performed during or after an initial capturing step. Purification can be performed using a resin, membrane, or any other solid support that binds either a therapeutic protein or contaminants (e.g., through the use of affinity chromatography, hydrophobic interaction chromatography, anion or cation exchange chromatography, or molecular sieve chromatography). A therapeutic protein can be purified from a fluid containing the therapeutic protein using at least one chromatography column and/or chromatographic membrane (e.g., any of the chromatography columns or chromatographic membranes described herein).

For example, immunoglobulin can be purified by the removal of contaminating non-immunoglobulin proteins; they are also purified by the removal of immunoglobulin other than IgG. The removal of non-immunoglobulin proteins and/or the removal of immunoglobulin other than IgG results in an increase in the percent of desired IgG in the feedstock. Purity can be measured by standard assays known in the art or described herein, examples of which include SDS-PAGE followed by Coomassie blue staining as well as chromatographic methods (e.g., size exclusion chromatography (SEC) on a HPLC system). Purity of the IgG sample can be calculated from an SDS PAGE gel after scanning, e.g., using a Kodak Image Station 1000 or equivalent system, or by analysis of SEC chromatogram by software on a Shimadzu HPLC system. A sample is considered pure if it is at least 90%, 95%, or 99% free of components other than the desired product (e.g., immunoglobulin).

Preferably, this method yields a preparation of Ig that is at least 80%, 85%, 90%, 95%, or 99% or more pure. “Pure” refers to a preparation of one or more Ig isotypes in which contaminating non-Ig proteins are reduced or it refers to a preparation of a first Ig isotype in which contaminating Ig of a second isotype is substantially absent. In an exemplary embodiment, the invention provides a preparation of an IgG subtype enriched in the subtype relative to a preparation of the subtype purified using only Protein A.

The term “capturing” means a step performed to partially purify or isolate (e.g., at least or about 5%, e.g., at least or about 10%, 15%, 20%, 25%, 30%, 40%, 45%, 50%, 55%, 60%, 65%, 70%, 75%, 80%, 85%, 90%, at least about 95% or at least about 99% pure by weight), optionally concentrate, and optionally stabilize a therapeutic protein from one or more other components present in a mixture containing the protein, e.g., an IgG-containing starting material. Typically, capturing is performed using a resin that binds a therapeutic protein (e.g., through the use of affinity chromatography). Non-limiting methods for capturing a therapeutic protein from a liquid are described herein and others are known in the art. A therapeutic protein can be captured from a liquid medium using at least one chromatography column and/or chromatographic membrane (e.g., any of the chromatography columns and/or chromatographic membranes described herein). In an exemplary embodiment, the invention provides a method of enriching a preparation in a selected Ig isotype or IgG subtype by capturing on an affinity column (other than Protein A) one or more isotype and/or subtype desired or other than that desired to be enriched.

The term “buffer” as used herein, refers to a substance which, by its presence in solution, increases the amount of acid or alkali that must be added to cause unit change in pH. A buffered solution resists changes in pH by the action of its acid-base conjugate components. Buffered solutions for use with biological reagents are generally capable of maintaining a constant concentration of hydrogen ions such that the pH of the solution is within a physiological range. Traditional buffer components include, but are not limited to, organic and inorganic salts, acids and bases.

The term “chromatography column” or “column” in connection with chromatography as used herein, refers to a container, frequently in the form of a cylinder or a hollow pillar which is filled with the chromatography matrix or resin. The chromatography matrix or resin is the material which provides the physical and/or chemical properties that are employed for purification.

The term “flow-through” or “flow-through mode” as used herein refers to the general purification approach wherein contaminants are removed from a mixture during chromatography because they are retained by a chromatographic process, usually bound to a chromatographic medium in a column. A protein of interest is purified because it does not bind (or it binds less strongly than contaminants) to a chromatographic medium, usually a chromatographic medium in a column, and instead flows through to be collected. “Flow-through” also refers to the liquid collected from this chromatographic mode. After passage of the liquid medium containing the protein of interest from the column, the impurities bound to the column are optionally “stripped”, or removed from the column, so that the column can then be regenerated for another chromatographic run. This approach differs from “bind-and-elute” or “bind-and-elute mode” wherein the target protein of interest is retained on the chromatographic medium within a column, and impurities flow through the column. This process then involves specific elution of the protein of interest using different column conditions that interfere with the binding of the protein of interest to the chromatographic medium, usually a resin in a column.

The term “polishing” is a term of art and means a step performed to remove remaining trace or small amounts of contaminants or impurities from a fluid containing a therapeutic protein that is close to a final desired purity. For example, polishing can be performed by passing a fluid containing the IgG through a chromatographic column(s) or membrane absorber(s) that selectively binds to either the IgG (target therapeutic protein) or small amounts of contaminants or impurities present in a fluid containing the target therapeutic protein. In such an example, the eluate/filtrate of the chromatographic column(s) or membrane absorber(s) contains the therapeutic protein.

As used herein the term “contaminant” or “impurity” is used in its broadest sense to cover any undesired component or compound within a mixture. Contaminant proteins include, without limitation, those naturally produced by a donor, or a host cell, as well as proteins related to or derived from the protein of interest (e.g., proteolytic fragments) and other process related contaminants. In certain embodiments, the contaminant precipitate is separated from the cell culture using another means, such as centrifugation, sterile filtration, depth filtration and tangential flow filtration. In various embodiments, a contaminant is an Ig isotype or and IgG subtype other than the isotype or IgG subtype desired to be enriched in a preparation by practicing a method of the invention to manufacture such a preparation. Exemplary contaminants depleted or removed during the disclosed process include one or more of Albumin, Apolipoprotein A-I, Complement C3, Haptoglobin, Fibrinogen, Complement C4-B, Complement C1s subcomponent, Complement C1r subcomponent, Apolipoprotein B-100, Alpha-2-macroglobulin. In an exemplary embodiment, these contaminants are removed during the on column solvent/detergent step.

The term “loading buffer” refers to the buffer used to prepare and load a mixture or other sample into the chromatography unit.

“Eluate,” as used herein, refers to a fluid that is emitted from a chromatography column or chromatographic membrane that contains a detectable amount of a protein.

“Therapeutic drug substance,” as used herein, refers to a substance including a protein, e.g., an IgG, that is sufficiently enriched, purified or isolated by a method of the invention from contaminating proteins, lipids, and nucleic acids (e.g., contaminating proteins, lipids, and nucleic acids present in a liquid culture medium or from a host cell (e.g., from a mammalian, yeast, or bacterial host cell) and biological contaminants (e.g., viral and bacterial contaminants)). An exemplary therapeutic drug substance can be formulated into a pharmaceutical agent without any further substantial purification and/or decontamination step.

The term “unit operation” refers to a functional step that can be performed in a process of the invention for manufacturing a therapeutic protein drug substance from a feedstock. For example, a unit operation can be filtering (e.g., removal of contaminant bacteria, yeast viruses, or mycobacteria, and/or particular matter from a fluid containing a therapeutic protein), capturing, chromatography, purifying, holding or storing, polishing, viral inactivating, adjusting the ionic concentration and/or pH of a fluid containing the therapeutic protein, and/or removing unwanted salts.

In some embodiments, the chromatographic system disclosed herein is on a skid. “Skid,” as used herein, refers to a three-dimensional solid structure that can act as a platform or support for a system described herein. A skid can, if it comprises one or more structures that enable movement (e.g., wheels, rollers, or the like), confer mobility on the system or a portion thereof.

The term “multi-column chromatography system” or “MCCS” means a system of a total of two or more interconnected or switching chromatography columns and/or chromatographic membranes. A non-limiting example of a multi-column chromatography system is a periodic counter current chromatography system (PCC) containing a total of two or more interconnected or switching chromatography columns and/or chromatographic membranes. Additional examples of multi-column chromatography systems are described herein and are known in the art. An exemplary MCCS of the invention includes a Protein A column fluidically linked to an affinity ligand column, which may itself be fluidically linked directly or indirectly to one or more additional affinity ligand columns.

In some embodiments, the methods and systems have a recovery from the feedstock of a desired protein, e.g., IgG or another selected Ig isoform, or an IgG subtype of at least about 40%, 50%, 55%, or 60%, and up to about 65%, 70%, 75%, 80%, 85%, 90%, 95%, 98% or at least about 99% of the amount of the protein contained in the feedstock loaded onto the first column. In some embodiments, the purification recovery is at least about 60% to about 70%. In an exemplary embodiment, the purification recovery is at least about 98% or at least about 99% of the amount of the protein contained in the feedstock loaded onto the first column. In various embodiments, the IgG purification recovery is at least about 98% or at least about 99% of the amount of the IgG contained in the feedstock loaded onto the first column. In an exemplary embodiment, the purification recovery of one or more of IgG1, IgG2, IgG3 and/or IgG4 is at least about 98% or at least about 99% of the amount of the relevant IgG contained in the feedstock loaded onto the first column.

As used herein, the term “mixing” describes an act of causing distribution of two or more distinct compounds or substances in a solution or suspension by any form of agitation. Complete equal distribution of all ingredients in a solution or suspension is not required as a result of “mixing” as the term is used in this application.

As used herein, the term “solvent” encompasses any liquid substance capable of dissolving or dispersing one or more other substances. A solvent may be inorganic in nature, such as water, or it may be an organic liquid, such as ethanol, acetone, methyl acetate, ethyl acetate, hexane, petrol ether, etc. As used in the term “solvent detergent treatment,” solvent denotes an organic solvent (e.g., tri-N-butyl phosphate), which is part of the solvent detergent mixture used to inactivate lipid-enveloped viruses in solution. An exemplary solvent/detergent process is performed on column and removes plasma protein contaminants, such as those noted above, in addition to virally derived contaminants.

As used herein, the term “detergent” is used in this application interchangeably with the term “surfactant” or “surface acting agent.” Surfactants are typically organic compounds that are amphiphilic, i.e., containing both hydrophobic groups (“tails”) and hydrophilic groups (“heads”), which render surfactants soluble in both organic solvents and water. A surfactant can be classified by the presence of formally charged groups in its head. A non-ionic surfactant has no charge groups in its head, whereas an ionic surfactant carries a net charge in its head. A zwitterionic surfactant contains a head with two oppositely charged groups. Some examples of common surfactants include: Anionic (based on sulfate, sulfonate or carboxylate anions): perfluorooctanoate (PFOA or PFO), perfluorooctanesulfonate (PFOS), sodium dodecyl sulfate (SDS), ammonium lauryl sulfate, and other alkyl sulfate salts, sodium laureth sulfate (also known as sodium lauryl ether sulfate, or SLES), alkyl benzene sulfonate; cationic (based on quaternary ammonium cations): cetyl trimethylammonium bromide (CTAB) a.k.a. hexadecyl trimethyl ammonium bromide, and other alkyltrimethylammonium salts, cetylpyridinium chloride (CPC), polyethoxylated tallow amine (POEA), benzalkonium chloride (BAC), benzethonium chloride (BZT); Long chain fatty acids and their salts: including caprylate, caprylic acid, heptanoat, hexanoic acid, heptanoic acid, nanoic acid, decanoic acid, and the like; Zwitterionic (amphoteric): dodecyl betaine; cocamidopropyl betaine; coco ampho glycinate; nonionic: alkyl poly(ethylene oxide), alkylphenol poly(ethylene oxide), copolymers of poly(ethylene oxide) and poly(propylene oxide) (commercially known as Poloxamers or Poloxamines), alkyl polyglucosides, including octyl glucoside, decyl maltoside, fatty alcohols (e.g., cetyl alcohol and oleyl alcohol), cocamide MEA, cocamide DEA, polysorbates (Tween 20, Tween 80, etc.), Triton detergents, and dodecyl dimethylamine oxide. In an exemplary embodiment, a detergent is combined with a solvent, and the combination utilized to perform a viral inactivation step on a protein preparation in a method of the invention. In various embodiments, this viral inactivation step occurs while a preparation of the invention is on a chromatography column utilized in a method of the invention.

“Protein A”, as used herein, refers to an affinity ligand, which is a 49 kDa surface protein originally found in the cell wall of the bacteria Staphylococcus aureus. This protein binds immunoglobulins, and comprises five homologous Ig-binding domains. Affinity chromatographic media including immobilized Protein A are known in the art. As will appreciated by those of skill in the art, other affinity ligands can be utilized in place of or in addition to Protein A, e.g., Protein G, Protein A/G and Protein L, in variations on the method disclosed herein, which are considered within the scope of the current invention. Those portions of the current disclosure expressly disclosing Protein A are also relevant to embodiments in which one or more of Protein G, Protein A/G and/or Protein L are utilized instead of or in addition to Protein A.

The term “does not appreciably elute”, as used herein, refers to a property of an aqueous solution, e.g., buffer, solvent/detergent, or other mixture when a protein immobilized on a chromatographic medium is contacted with the solution, namely, that either none or an only very small amount of the protein is eluted off of the chromatographic medium by the solution. In an exemplary embodiment, less than about 5%, less than about 3%, less than about 1%, or less than about 0.1% of the protein is eluted from the chromatographic medium by the solution. An exemplary solution that does not appreciably elute the protein is a solvent/detergent solution. An exemplary protein not eluted from the chromatographic medium by the solvent/detergent solution is IgG. In an exemplary embodiment, “does not appreciably elute” refers to less than about 5% of the IgG immobilized on a chromatographic medium is eluted from the chromatographic medium by contact with a solvent/detergent solution.

A table entry herein bearing the notation “x” indicates that the relevant component has not been analyzed.

### C. Abbreviations

BDS, bulk drug substance; CRS, cryo supernatant (supernatant after first precipitation in the COHN-fractionation); CV, column volume; DFM, data file management (protocol system); E, Eluate fraction; EDTA, ethylenediaminetetraacetic acid; EtOH, Ethanol; Fc, antibody FC domain; FT, flow through; HPLC, high-performance liquid chromatography; IgG, immune gamma globulin; IgG1, IgG2, IgG3, IgG4, immune gamma globulin subclass 1-4; IQEQ, equilibration buffer (an exemplary IQEQ allows further purification of plasma proteins from the flow through of one or more affinity column after absorption of the IgG; e.g., 10 mM NaAcetate, 10 mM TrisHCl, 120 mM NaCl, pH 7.2); MAB, monoclonal antibody; MWCO, molecular weight cut off; SD_VI, solvent detergent-virus inactivation, e.g., 10 mM NaAcetate, 10 mM TrisHCl, 120 mM NaCl with SD_VI_MIX Triton X 10.55 g/Polysorbate 80 3.21 g/TnBP 2.91 g per kg buffer); SEC, size exclusion chromatography; TnBP, tri-N-butylphosphate; vWF, von-Willebrand-Factor; W, wash fraction; WB, western blot; WSD, wash solvent/detergent.

### C. The Embodiments

In an exemplary embodiment, the present invention is directed to an affinity chromatographic method of preparing from a feedstock e.g., an IgG-containing starting material, an IgG formulation comprising one or more of IgG1, IgG2, IgG3 and IgG4. The method includes (a) contacting the feedstock with a first chromatographic medium comprising a first solid support having Protein A bound thereto, forming a Protein A bound fraction of an IgG subclass with Protein A binding affinity and forming a first flow through comprising an IgG subclass not binding to the Protein A; (b) contacting the first flow through with a first affinity ligand chromatographic medium comprising a second solid support having a first affinity ligand with IgG binding affinity bound thereto, forming a first affinity ligand-bound IgG subclass and a second flow through comprising an IgG subclass not bound to the first affinity ligand-bound IgG subclass; (c) eluting the Protein A bound fraction from the first chromatographic medium, forming a first eluate and contacting the first eluate with the first affinity ligand chromatographic medium, forming a second affinity ligand-bound IgG subclass, and a second flow through; and (d) eluting a member selected from the first affinity ligand-bound IgG subclass, the second affinity ligand-bound IgG subclass, and a combination thereof from the first affinity ligand column, forming a second eluate.

In an exemplary embodiment, the method further includes, (e) combining the first flow through and the second eluate on the first affinity ligand chromatographic medium.

In an exemplary embodiment, the method includes (f) contacting the second eluate of (d) with a second affinity ligand chromatographic medium comprising a third solid support having a second affinity ligand with IgG binding affinity bound thereto, forming a third affinity ligand-bound IgG subclass and a second flow through comprising a fourth IgG subclass not bound to the second affinity ligand chromatographic medium; and (g) eluting a member selected from the second affinity ligand bound IgG subclass, the fourth IgG subclass and a combination thereof from the second affinity ligand chromatographic medium, forming a third eluate.

In an exemplary embodiment, the method includes (h) prior to (g), combining the first flow through and the second eluate on the second affinity ligand chromatographic medium.

In various embodiments, the method includes (i) prior to (b), contacting the first chromatographic medium with a buffer/detergent mixture that does not elute the Protein A bound fraction of an IgG subclass with Protein A binding affinity to from the first chromatographic medium.

In some embodiments, the method includes (j) prior to (g), contacting the second affinity ligand chromatographic medium with a buffer/detergent mixture that does not elute the second affinity ligand-bound IgG subclass from the second affinity ligand chromatographic medium.

An exemplary embodiment includes dedicated washing steps and an on-column solvent/detergent, viral inactivation step (“On column” SD_VI treatment), thereby reducing the total number of unit operations compared to traditional IgG fractionation and purification. The on-column SD_VI step can be performed on any column or more than one column within the array. In various embodiments, the on-column SD_VI further enhances the efficiency and compactness of the inventive process, particularly relative to art-standard fractionation procedures, by incorporating the SD_VI step directly into an established unit operation as opposed to it being conducted as a stand-alone step on material separated from the chromatographic medium. In fact, further unexpected was the enhanced purity of IgG formulations in which the on-column SD_VI step was incorporated into the purification as this step was not contemplated to have an effect beyond viral inactivation. The SD_VI step depletes or removes impurities other than those virally derived.

In various embodiments, the SD_VI step is performed following an IQEQ step in which the target therapeutic protein (e.g., IgG) is captured on the chromatographic medium and a flow through is produced, and prior to eluting the target therapeutic protein from the chromatographic medium.

In exemplary embodiments, the SD_VI step depletes or removes at least one of Albumin, Apolipoprotein A-I, Complement C3, Haptoglobin, Fibrinogen, Complement C4-B, Complement C1s subcomponent, Complement C1r subcomponent, Apolipoprotein B-100, Alpha-2-macroglobulin. In an exemplary embodiment, these impurities are found in the wash from the SD_VI step.

In an exemplary embodiment, the buffer/detergent mixture includes acetate, Tris, Triton X, tri(n-butyl)phosphate (TnBP).

In an exemplary embodiment, the buffer/detergent mixture includes a buffer component having about 10 mM sodium acetate, about 10 mM TrisHCl, and about 120 mM NaCl, and a detergent component having about 10.55 g Triton X, 3.21 g Polysorbate 80 and about 2.91 g TnBP per kg of the buffer solution.

The method and systems of the invention provide formulations in which IgG content is significantly enriched relative to the feedstock.

In an exemplary embodiment, IgG in the formulation is at least about 90% wt/wt of the protein content of the IgG formulation of the invention.

In an exemplary embodiment, IgG1 in the formulation is about 40% to about 90% wt/wt of the protein content of the formulation.

In an exemplary embodiment, IgG2 in the formulation is about 15% to about 55% wt/wt of the protein content of the formulation.

In an exemplary embodiment, IgG3 in the formulation is about 0.5% to about 50% wt/wt of the protein content of the formulation.

In an exemplary embodiment, IgG4 in the formulation is about 0.1% to about 20% wt/wt of the protein content of the formulation.

Exemplary methods of the invention provide a highly pure formulation of IgG. In various embodiments, the invention provides a highly pure formulation of one isoform of IgG.

In an exemplary embodiment, albumin in the formulation is in an amount less than about 5 g/L, e.g., less than about 2 g/L, less than about 1 g/L, less than about 0.4 g/L, less than about 0.2 g/L, less than about 0.1 g/L, or less than about 0.05 g/L of the formulation.

In an exemplary embodiment, Factor XI activity in the formulation is less than about 0.01 E/mLt, e.g., less than about 0.005 E/mLt, e.g., less than about 0.001 E/mLt.

In an exemplary embodiment, alpha-2-macroglobulin present in the formulation is in an amount less than about 0.2 g/L, e.g., less than about 0.1 g/L, e.g., less than about 0.05 g/L of the formulation.

In an exemplary embodiment, transferrin present in the formulation is in an amount less than about 0.4 g/L less than about 0.2 g/L, e.g., less than about 0.09 g/L of the formulation.

In an exemplary embodiment, PL1 activity of the formulation is less than about 10 nm/mL min.

In an exemplary embodiment, fibrinogen present in the formulation is in an amount less than about 60 μg/mL, less than about 35 μg/mL, e.g. less than about 13 μg/mL of the formulation.

In an exemplary embodiment, the IgG-containing starting material is a cryosupernatant.

In an exemplary embodiment, a member selected from the first chromatographic medium comprising a first solid support having Protein A, the first affinity ligand chromatographic medium comprising a second solid support having a first affinity ligand with IgG binding affinity bound thereto, and a combination thereof is contained in an individual column.

In an exemplary embodiment, the invention provides a system and a method of using the system in which two or more of chromatographic columns containing protein A, one or more of the affinity ligand chromatographic media and a combination thereof are in fluidic communication within an array of columns, and two or more of the columns comprise a second chromatographic medium and a second affinity ligand chromatographic medium. The columns are optionally fluidically coupled by means of fluid inlets, fluid outlets and tubing joining one or more outlet from one or more column to one or more inlet for one or more column. The columns in the array can fluidically communicate in series, in parallel or in a combination of these configurations.

In an exemplary embodiment, the outlet of a first column is in fluidic communication with the inlet of a second column, allowing the eluate from the first column to load directly into the inlet of the second column, and hence onto the chromatographic medium contained therein.

In an exemplary embodiment, the outlet of the column having Protein A is in fluidic communication with the inlet of the first affinity ligand chromatographic medium.

In an exemplary embodiment, the outlet of a column containing a second chromatographic medium having bound thereto a first affinity ligand is in fluidic communication with the inlet of a column containing a second affinity ligand chromatographic medium.

In various embodiments, the outlet of a first column switchably communicates with the inlet of one or more second columns. In an exemplary embodiment, the outlet(s) of one or more first columns switchably communicates with the inlet(s) of one or more second columns.

In an exemplary embodiment, the IgG solution depleted in a Protein A binding IgG subclass includes a member selected from IgG1, IgG2, IgG3, IgG4 and a combination thereof.

In another an exemplary embodiment, the first chromatographic medium has selective affinity for the CH1 domain of human IgG antibodies.

In an exemplary embodiment, the first chromatographic medium has selective affinity for the CH1 domain of a member selected from IgG1 and IgG3, which is greater than the selective affinity for the CH1 domain of a member selected from IgG2 and IgG4.

In an exemplary embodiment, eluting from the first chromatographic medium, the first affinity ligand chromatographic medium, and/or the second affinity ligand chromatographic medium utilizes an eluent comprising glycine.

In an exemplary embodiment, the glycine concentration is from about 50 mM to about 150 mM. In an exemplary embodiment, the glycine concentration is about 130 mM.

Other eluents are of use in the present invention including, without limitation, citrate, acetate, MES, HCl at pH below 4.0 and combinations thereof.

An exemplary embodiment of the present invention is directed to an affinity chromatographic method of preparing an IgG formulation enriched in at least a first IgG subclass with low protein A binding affinity from a plasma-derived IgG solution depleted in a Protein A binding IgG subclass. The method includes (a) contacting the IgG solution depleted in a Protein A binding IgG subclass with a first affinity ligand chromatographic medium comprising a first solid support having a first affinity ligand with IgG binding affinity bound thereto, forming a first bound fraction of the first IgG subclass with low protein A binding affinity and a first flow through; and (b) eluting the first bound fraction of the first IgG subclass with low protein A binding affinity, forming a first eluent comprising the IgG formulation enriched in the first IgG subclass with low protein A binding.

An exemplary-derived IgG solution depleted in a Protein A binding IgG subclass is prepared by passing an IgG-containing starting material through a chromatographic medium comprising Protein A, binding the IgG subclass or subclasses with Protein A affinity, and collecting from the chromatographic medium, generally in the flow through, those IgG subclasses without sufficient affinity to Protein A to remain bound to Protein A during this process.

In an exemplary embodiment, the method further includes (c) prior to (b), contacting the first bound fraction of the first IgG subclass with low protein A binding affinity with a buffer/detergent solution, and subsequently removing the detergent solution from the first affinity ligand chromatographic medium.

In an exemplary embodiment, the method further includes (d) following (b), contacting the first eluent with a second chromatographic medium comprising a solid support with an affinity ligand having affinity for a second IgG subclass with low protein A binding affinity bound thereto, forming a first bound fraction of a second IgG subclass with low protein A binding affinity; and (e) eluting from the second chromatographic medium the first bound fraction of the second IgG subclass with low protein A binding affinity, forming a second eluate.

In some embodiments, the method further includes (f) prior to (e), contacting the first bound fraction of the second IgG subclass with low protein A binding affinity with a buffer/detergent solution, and subsequently removing the detergent solution from the first chromatographic medium.

In various embodiments, the method further includes (g) prior to (a), contacting a plasma-derived IgG solution with a Protein A chromatographic medium comprising Protein A bound to a solid support, forming a Protein A-bound fraction of an IgG subclass with high affinity for Protein A, and the IgG formulation enriched in a first IgG subclass with low protein A binding affinity.

In an exemplary embodiment, the method further includes (h) eluting the Protein A-bound fraction of an IgG subclass with high affinity for Protein A from the Protein A chromatographic medium, forming a third eluate.

An exemplary method includes a step of combining two or more of the first eluate, the second eluate and the third eluate.

An exemplary method of the invention uses two, three or more columns in series. The starting protein mixture is applied to the inlet of the first column (e.g., Protein A) and then a pre-selected volume of a wash solution is passed through the column, removing unbound IgG, and impurities and, in some embodiments, progressing a population of the proteins in the mixture through the first column and into one or more of the succeeding columns. Flow through exiting the terminal column is optionally collected. Following the wash cycle, an eluent, in some embodiments, a slightly acidic eluent, is passed into the inlet of the first column, eluting a population of protein bound to the chromatographic medium into the second column, a population of the proteins bound to the chromatographic medium in the second column through the terminus of the apparatus or, in some embodiments, into a third column packed with a third chromatographic medium, and so on. In the elution step, a population of proteins bound to the chromatographic medium (e.g., second affinity ligand medium) in the first column is desorbed from this medium, pass into the second column and become bound to the second chromatographic medium in the second column. The same is true of proteins adsorbed onto the second medium, which are desorbed and pass into a third column in embodiments in which a third column is present, are optionally collected at the terminus of the second column in those embodiments in which a third column is not present, or are collected at the terminus of the third column. The process described above is optionally repeated for the third and subsequent columns.

In an exemplary embodiment, the flow through and/or eluate from each column or a subset of the columns can be collected separately or pooled before being loaded onto a succeeding column. Embodiments in which a combination of serial columns and one or more individual column(s) are utilized are also within the scope of the present invention.

Also provided herein is an IgG formulation prepared by a method of the invention. In an exemplary embodiment, the formulation has an IgG subtype profile essentially identical to that of human plasma. In an exemplary embodiment, the formulation has an impurity profile essentially identical to that of human plasma. In an exemplary embodiment, the formulation has both an IgG subtype profile and an impurity profile essentially identical to that of commercial polyclonal IgG formulations, e.g., Gammagard liquid, Kiovig, Cuvitru.

The aqueous IgG solution according to the invention can be further processed by one or more ion exchange steps and by formulating it into a final pharmaceutically acceptable formulation.

In an exemplary embodiment, there is provided an aqueous IgG solution comprising at least about 99.5% IgG and less than about 0.5% impurities whereby the aqueous IgG solution comprises Factor XI as an impurity at an activity of less than about 0.0025 E/mg of total IgG, preferably less than about 0.0020 E/mg of total IgG, more preferably less than about 0.0015 E/mg of total IgG, and most preferably less than about 0.0012 E/mg of total IgG.

In an exemplary embodiment, there is provided an aqueous IgG solution comprising at least about 99.5% IgG and less than about 0.5% impurities whereby the aqueous IgG solution comprises Complement C3 as an impurity at a level of less than about 3.5 μg/mg of total IgG, preferably less than about 3.0 μg/mg of total IgG, more preferably less than about 2.5 μg/mg of total IgG, and most preferably less than about 2.2 μg/mg of total IgG.

In an exemplary embodiment, there is provided an aqueous IgG solution comprising at least about 99.5% IgG and less than about 0.5% impurities whereby the aqueous IgG solution comprises as an impurity Complement C3 and Factor XI, whereby Complement C3 is present at a level of less than about 3.5 μg/mg of total IgG, preferably less than about 3.0 μg/mg of total IgG, more preferably less than about 2.5 μg/mg of total IgG, and most preferably less than about 2.2 μg/mg of total IgG and whereby Factor XI is present at an activity of less than about 0.0025 E/mg of total IgG, preferably less than about 0.0020 E/mg of total IgG, more preferably less than about 0.0015 E/mg of total IgG, and most preferably less than about 0.0012 E/mg of total IgG. In an exemplary embodiment, there is provided an aqueous IgG solution comprising at least about 99.5% IgG and less than about 0.5% impurities whereby the aqueous IgG solution comprises as an impurity Complement C3 and Factor XI, whereby Complement C3 is present at a level of less than about 2.5 μg/mg of total IgG and whereby Factor XI is present at an activity of less than about 0.0015 E/mg of total IgG.

The following examples are offered to illustrate certain exemplary embodiments of the invention, and are not to be construed as limiting the scope of the invention in any manner. Affinity purification methods were evaluated for the ability to develop next generation human IgG products equal or superior to current available IgG products. To date, affinity purification has not been used on a large scale to replace fractionation to purify IgG or other proteins from an IgG-containing starting material.

### EXAMPLES

### Example 1

### 1.1 Experimental Overview

Initial experiments were performed with a purified plasma derived polyclonal IgG as starting material. The aim was to quickly gather information with a defined IgG product, already used in clinical application, as a benchmark.

Within the first screening several potential candidates for the second affinity purification step were evaluated as stand-alone chromatography runs in parallel. Further on a tandem approach was performed as described below:
1. Capture of the main proportion of IgG with a Protein A derived resin
2. Capture of the residual IgG present in the flow through (FT) of the Protein A capture with an alternative IgG binding affinity resin

In a next step the resin identified with the best binding properties (capacity) was chosen for an additional experimental setup. In this approach the capture of IgG out of cryo supernatant was evaluated.

The first set of experiments were designed for the screening of combinations of Protein A and resins in a “Tandem”-array to capture a full spectrum of IgG subclasses. As starting material, a liquid purified plasma derived polyclonal IgG preparation was used.

The second set of experiments were designed to optimize the IgG subclass spectrum directly with modified Cohn cryo-supernatant as starting material.

### 1.2 Biological Materials

1. Purified plasma derived polyclonal IgG
2. Cryo-supernatant

### 1.3 Buffers

All buffers used for the experiments were prepared at the operating department. The buffers were stored at +2 to +8° C.

### 1.4 Selection of Resins

For the direct capture Mab select PrismA (GE Healthcare) was selected due the high resistance against sodium hydroxide.

Cryo supernatant contains all blood coagulation factors except FVIII and vWF, which are reduced after cryo precipitation. This fact can potentially lead to partial clotting and precipitation on the resin, thus an efficient regeneration and cleaning procedure is required.

The resins selected for the capture of the residual IgG sub-population are commercially available chromatography resins provided by Thermo Fisher.

TABLE 1
List of Resins Screened for Improved IgG Capture
Resins

1. CaptureSelect ™ FcXL Affinity Matrix
2. CaptureSelect ™ CH1-XL Affinity Matrix
3. CaptureSelect ™ KappaXL Affinity Matrix
4. CaptureSelect ™ IgG-Fc (ms) Affinity Matrix
5. CaptureSelect ™ LC-lambda (Hu) Affinity Matrix
6. CaptureSelect ™ KappaXP Affinity Matrix
7. CaptureSelect ™ IgG3 Affinity Matrix*
*Not applied in the screening phase due to the fact that Capture Select CH1-XL, FcXL and FcMS were able to bind ≥97% of IgG3 present in purified polyclonal IgG solution.

### 1.5 Evaluation of Single Unit Operations/Clusters

TABLE 2
Buffers Used During Screening Phase

| Nr.: | Buffer ID | Buffer composition | pH | Name |
|---|---|---|---|---|
| 1. | Equilibration/ | 10 mM NaAcetate, 10 mM | 7.2 | IQEQ |
| | Dilution buffer | TrisHCl, 120 mM NaCl | | |
| 2. | Wash/SD_VI | 10 mM NaAcetate, 10 mM | 7.2 | IQEQ_VI |
| | On Column | TrisHCl, 120 mM NaCl with | | |
| | buffer | SD_VI_MIX Triton X 10.55 | | |
| | | g/Polysorbate 80 3.21 g/TnBP | | |
| | | 2.91 g per kg buffer | | |
| 3. | Elution buffer 1 | 100 mM Glycine | 3.2 | E1 |
| 4. | Elution buffer 2 | 100 mM Glycine | 2.7 | E2 |

### 1.6 Chromatography Scheme for Phase 1 (Prisma Affinity)

A chromatography column with the following dimensions was used for the Protein A capture step:
Column: 3.2 cm h=7.5 cm A=8.04 cm² V=60.3 ml

TABLE 3
Chromatography Scheme IG_MAX

| | | Inlet | | Flowrate | Outlet | |
|---|---|---|---|---|---|---|
| Chromatography Step | Buffer/Sample | Valve | CV | (ml/min) | valve | Fraction |
| Equilibration | IQEQ | A1 | 2 | 10 | — | Waste |
| Resin activation | 100 mM GlycinE pH | B1 | 3 | 10 | — | Waste |
| | 2.7 | | | | | |
| Equilibration | IQEQ | A1 | 5 | 10 | — | Waste |
| Sample application | — | S1 | mL | 10 | 1 | FT |
| WASH 1 Re- | IQEQ | A1 | 2 | 10 | 1 | FT |
| equilibration | | | | | | |
| SD_VI ON Column | IQEQ_VI | A2 | 4 | 4.0 | 2 | WSD |
| WASH 2 | IQEQ | A1 | 10 | 10 | 3 | W2 |
| Elution 1 | E1 | B1 | 4 | 5 | 4 | E1 |
| Elution 2 | E2 | B2 | 4 | 5 | 5 | E2 |
| Reconditioning | IQEQ | A1 | 4 | 10 | — | Waste |

### 1.7 Results of the Screening Phase/Experiments

a. Affinity of Purified IgG-Containing Starting Material on MAb Select PrismA

The chromatography runs on the Protein A capture column were performed as previously outlined in the chromatography scheme for phase 1 with a calculated column load of 30 mg IgG per ml of resin.

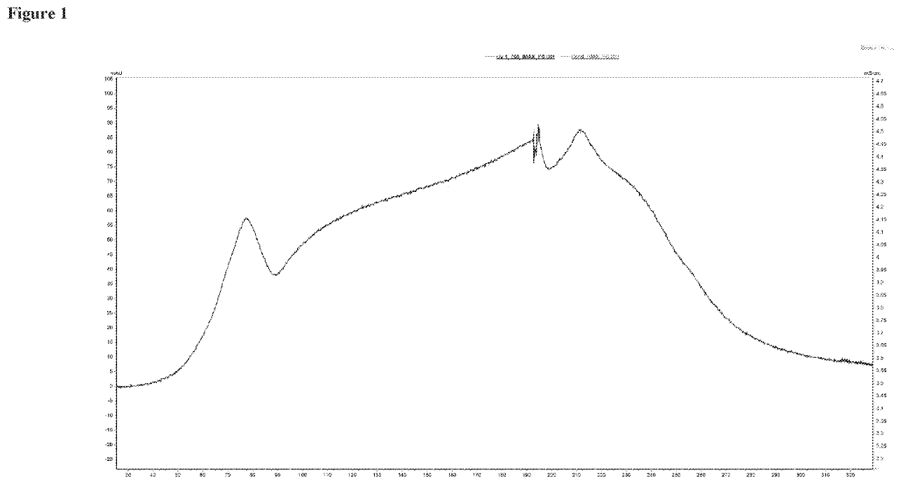

In FIG. 1, the flow through UV profile is shown. The low extinction gives information about the high efficiency of binding of human IgG in this experimental setup using IgG-containing starting material.

Flow through fraction (UV 280 signal) of the PrismA run using IgG-containing starting material.

This flow through fraction is collected and used as load for further screening of affinity resins with different specificities for immunoglobulin subpopulations as described in the selection of resins.

### 1.8 IgG Content and Subclass Distribution of the Load and Flow Through (FT) Fractions of MabSelect PrismA Resin

TABLE 4
Run IGR01: IgG1, IgG2, IgG3 and IgG4
in the “Flowthrough (FT)”

| | IgG1 | IgG2 | IgG3 | IgG4 |
|---|---|---|---|---|
| LOAD [mg] | 1218.5 | 547.1 | 52.03 | 65.42 |
| FT [mg] | 18.6 | <0.1 | 33.4 | <0.1 |
| % in FT ² | 1.5 | <0.1 | 64.2 | <0.1 |

¹ Ratio based wherein IgG3 is 1 part/Ratio are given as the x-fold part of IgG3
² Percent of IGG subclasses found in the PrismA Flow through
Only IgG3 is found in significant amounts in the flow through of Mab Select PrismA.

### Example 2

### 2.1 Chromatography Scheme for Phase 2 (Resin Screening)

A chromatography column with the following dimensions was used for the resin screening runs using Protein A capture flow through:
Column: 10 cm h=3 to 4 cm A=0.79 cm² V=3.5 to 4.5 ml

The chromatographic procedure for these runs is listed under Chromatography Procedure for the Resin Screening Run.

### 2.2 Chromatography Procedure for the Resin Screening Run

TABLE 5
Chromatography Scheme for Resin Screening

| | Buffer/ | Inlet | | Flowrate | Outlet | |
|---|---|---|---|---|---|---|
| Chromatography Step | Sample | Valve | CV | (ml/min) | valve | Fraction |
| Equilibration | IQEQ | A1 | — | 0.30 | — | Waste |
| Resin activation | E2 | B1 | 5 | 0.30 | — | Waste |
| Equilibration | IQEQ | A1 | 10 | 0.30 | — | Waste |
| Sample application | Variable 1 | S1 | 45-50 | 0.30 | 1 | FT |
| | | | mL | | | |
| Wash 1 Re- | IQEQ | A1 | 5 | 0.30 | 1 | FT |
| equilibration | | | | | | |
| SD_VI ON Column | IQEQ_VI | A2 | 10 | 60 min | 2 | WSD |
| Wash 2 | IQEQ | A1 | 20 | 0.79 | 3 | W2 |
| Elution 1 | E1 | B1 | 10 | 0.30 | 4 | E1 |
| Reconditioning | IQEQ | A1 | 10 | 0.79 | — | Waste |

### 2.3 Capture Select Flow Through Evaluation UV 280 nm Signal

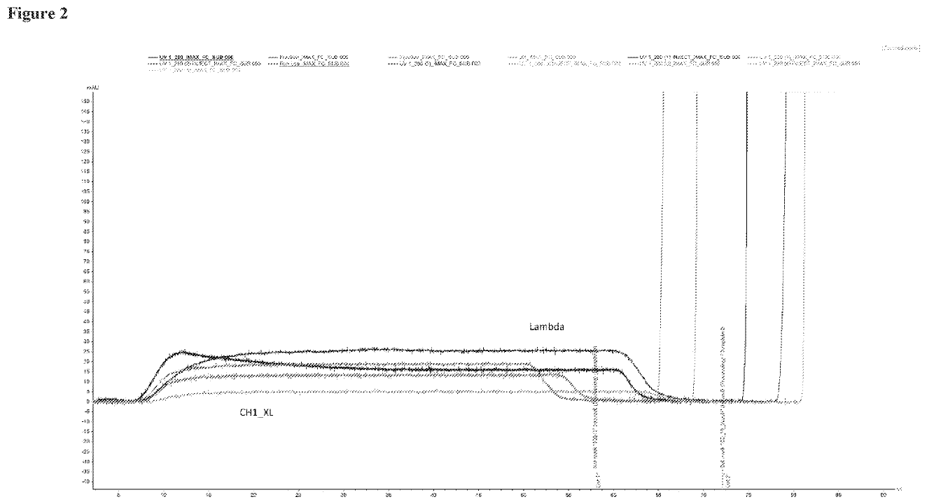

FIG. 2 shows the flow through profiles of the screened resin. Different UV 280 signal heights were observed for e.g. anti-Lamba or anti-CH1-XL. Lower flow through UV levels led to higher UV elution peaks.

### 2.4 Capture Select Eluates Evaluation UV280 nm Signal

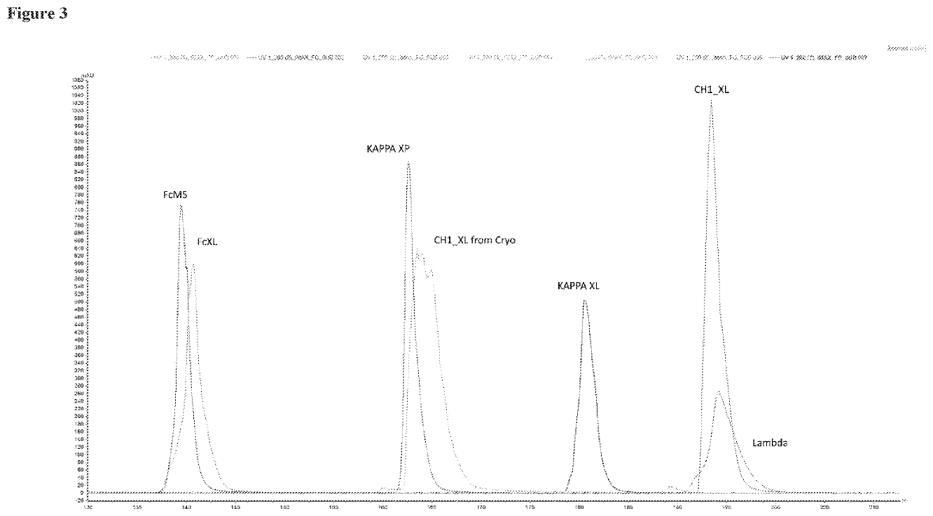

FIG. 3 shows the elution profiles of the screened resin. Different UV280 signal heights and areas were observed. Capture Select CH1-XL has shown the highest peak and the largest peak area.

Based on these data a ranking of the CaptureSelect resins was performed. The most efficient resin was CaptureSelect CH1-XL.

### 2.5 Data Evaluation

a. Summary of Peak Areas of Capture Select Affinity Eluates of PrismA Flow Through of Purified IgG-Containing Starting Material

Table 6 shows the peak areas (UV280 nm) of the different affinity columns tested. Analytical results of the Protein A resin are listed under IgG content and subclass distribution of the load and flow through (FT) fractions of MabSelect PrismA resin. IgG2 and IgG4 are captured almost quantitatively, IgG1 and IgG3 to a lesser efficiency. These subtypes are captured by the second resin. CaptureSelect CH1-XL was identified to bind the main amount of the residual IgG subspecies out of Mab Select PrismA flow through. This resin was introduced as a second column to run the next experiments.

TABLE 6
Evaluation of Chromatograms (Peak Areas)

| | | | | Peak |
|---|---|---|---|---|
| Resins for | | Outcome/ | Peak | max. |
| column II | Run ID | ranking | AREA | (280 nm) |
| CaptureSelect ™ | FcXL | 3 | 1100 | 600 |
| FcXL | | | | |
| CaptureSelect ™ | CH1_XL | 1 | 1909 | 1021 |
| CH1-XL | | | | |
| CaptureSelect ™ | KappaXL | 5 | 972 | 504 |
| KappaXL | | | | |
| CaptureSelect ™ | FcMS | 4 | 1037 | 752 |
| IgG-Fc (ms) | | | | |
| CaptureSelect ™ | Lambda | 6 | 865 | 266 |
| LC-lambda (Hu) | | | | |
| CaptureSelect ™ | Kappa_XP_P | 2 | 1192 | 865 |
| KappaXP | | | | |
| Affinity | | | | |

### Example 3

### 3.1 Evaluation of the Capture Select Resins for Binding of the Remaining IgG Subclasses in Mab Select PrismA Flow Through

In the following tables the recoveries of IgG1 and IgG3 out of the flow-through fraction of Mab select PrismA with the different Capture Select columns are summarized. Only the flow through and the elution fractions are listed. Portions may be in the flow through fractions (below detection limit) and in wash and post-elution fractions (not analyzed).

TABLE 7
Capture Select CH1 XL Run Nr.: CH1XL

| | IgG1 | IgG1 | IgG3 | IgG3 |
|---|---|---|---|---|
| LOAD | 2.97 mg | 100% | 5.33 mg | 100% |
| FT | Below | n.a | Below | n.a |
| | detection limit | | detection limit | |
| ELUATE | 2.54 mg | 85.4% | 3.77 mg | 70.8% |

n.a = not applicable

TABLE 8
Capture Select KAPPA XL Run Nr.: KAPPA XL

| | IgG1 | IgG1 | IgG3 | IgG3 |
|---|---|---|---|---|
| LOAD | 2.38 mg | 100% | 4.27 mg | 100% |
| FT | Below | n.a | 0.94 mg | 22.1% |
| | detection limit | | | |
| ELUATE | Below | n.a | 1.28 mg | 30.0% |
| | detection limit | | | |

n.a = not applicable

TABLE 9
Capture Select LC_LAMBDA(Hu)Run Nr.: LAMBDA(Hu)

| | IgG1 | IgG1 | IgG3 | IgG3 |
|---|---|---|---|---|
| LOAD | 2.88 mg | 100% | 5.17 mg | 100% |
| FT | Below | n.a | 1.88 mg | 36.3% |
| | detection limit | | | |
| ELUATE | 1.66 mg | 57.7% | 1.19 mg | 22.9% |

n.a = not applicable

TABLE 10
Capture Select FcXL Run Nr.: FcXL

| | IgG1 | IgG1 | IgG3 | IgG3 |
|---|---|---|---|---|
| LOAD | 2.52 mg | 100% | 4.52 mg | 100% |
| FT | Below | n.a | 0.02 mg | 2.8% |
| | detection limit | | | |
| ELUATE | 2.17 mg | 86.0% | 2.94 mg | 65.0% |

n.a = not applicable

TABLE 11
Capture Select KAPPA XP Run Nr.: KAPPA_XP_P

| | IgG1 | IgG1 | IgG3 | IgG3 |
|---|---|---|---|---|
| LOAD | 2.90 mg | 100% | 5.20 mg | 100% |
| FT | Below | n.a | 1.53 mg | 29.4% |
| | detection | | | |
| | limit | | | |
| ELUATE | 2.96 mg | 102.0% | 2.89 mg | 55.7% |

n.a = not applicable

TABLE 12
Capture Select FcMS Run Nr.: FcMS

| | IgG1 | IgG1 | IgG3 | IgG3 |
|---|---|---|---|---|
| LOAD | 2.37 mg | 100% | 4.25 mg | 100% |
| FT | Below | n.a | 0.12 mg | 2.8% |
| | detection | | | |
| | limit | | | |
| ELUATE | 1.91 mg | 80.7% | 2.88 mg | 67.8% |

n.a = not applicable

### Example 4

### 4.1 Variant IGC02

a. Buffer Compositions Used in IGC02

TABLE 13
Buffer Compositions

| Buffer | Buffer ID | Buffer composition | pH | Name |
|---|---|---|---|---|
| 1. | Equilibration/ | 10 mM NaAcetate, | 7.2 | IQEQ |
| | Dilution buffer | 10 mM TrisHCl, | | |
| | | 120 mM NaCl | | |
| 2. | WASH/SD_VI | 10 mM NaAcetate, | 7.2 | IQEQ_VI |
| | On Column | 10 mM TrisHCl, | | |
| | buffer | 120 mM NaCl + | | |
| | | SD_VI_MIX Triton | | |
| | | X 10.55/Polysorbate | | |
| | | 80 3.21/TnBP 2.91 | | |
| 3. | Elution buffer 1 | 100 mM Glycine | 3.2 | E1 |
| 4. | Elution buffer 2 | 100 mM Glycine | 2.7 | E2 |

### 4.2 IGC02 Run

IGC02 was carried out in 2 steps—at first the affinity on MAB Select PrismA was performed and as a second step the MAB Select PrismA flow through was applied onto Capture Select CH1 XL.

Cryo-supernatant from the Cohn fractionation process was introduced into the purification process without any further treatment prior to the experiment.

a. First Step

After the equilibration of the MAB-Select PrismA (GE Healthcare, Cat.Nr.: 17-5498-02, ID: 3.2 cm h=7.5 cm A=8.04 cm² V=60.3 ml) with IQEQ buffer the cryo-supernatant was applied onto the column with a contact time of approx. 6 min.

After the load, a first wash was applied with 2 CV of buffer IQEQ to remove unbound proteins from the resin (added to flowthrough). A SD_VI on column treatment was carried out by applying IQEQ_VI buffer which contains the detergent composition of 10.55 g Triton X-100, 3.21 g Polysorbate 80 and 2.91 g TnBP in 1 kg of IQEQ buffer (corresponding to 1%/0.3%/0.3% of Triton X100/Polysorbate 80/TnBP). The SD_VI on column is run by contacting the bound proteins for at least about 60 minutes with IQEQ_VI buffer with a flow rate of 4 ml/min within 4 CV. The wash is optionally collected for analysis.

Wash 2 was carried out with IQEQ buffer for further depletion of impurities and to wash out the SD_VI reagents in 10 CV's and a flow rate of 10 ml/min. The wash is optionally collected for analysis.

The elution was performed by applying 5 CV's of E1 buffer (100 mM Glycine, pH 3.2). The 1^{st} step of a cleaning procedure (Strip) was carried out with E2 buffer (100 mM Glycine, pH 2.7).

After a re-equilibration of 4 CV of IQEQ buffer the column was regenerated and stored.

Regeneration procedure and storage are described under Regeneration Procedure.

### Example 5

### 5.1 Column

MAB Select PrismA
GE Healthcare, Cat.Nr.: 17-5498-02
Lot.Nr.: 10264190
ID: 3.2 cm h=7.5 cm A=8.04 cm² V=60.3 ml (Target)

TABLE 14
Chromatography Scheme

| | | Inlet | | Flowrate | Outlet | |
|---|---|---|---|---|---|---|
| Chromatography Step | Buffer/Sample | Valve | CV | (ml/min) | valve | Fraction |
| Equilibration | IQEQ | A1 | 2 | 10 | — | Waste |
| Resin activation | 100 mM Glycine, pH 2.7 | B1 | 3 | 10 | — | Waste |
| Equilibration | IQEQ | A1 | 5 | 10 | — | Waste |
| Sample application | — | S1 | mL | 10 | 1 | FT |
| WASH 1 Re-equilibration | IQEQ | A1 | 2 | 10 | 1 | FT |
| SD_VI On Column | IQEQ_VI | A2 | 4 | 4.0 | 2 | WSD |
| WASH 2 | IQEQ | A1 | 10 | 10 | 3 | W2 |
| Elution 1 | E1 | B1 | 4 | 5 | 4 | E1 |
| Elution 2 | E2 | B2 | 4 | 5 | 5 | E2 |
| Reconditioning | IQEQ | A1 | 4 | 10 | — | Waste |

a. Chromatogram IGC02

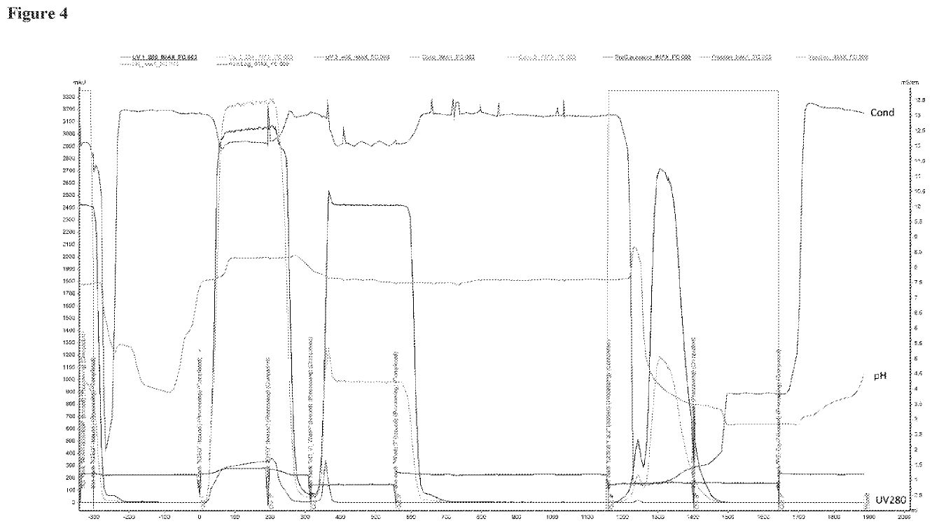

See FIG. 4.

### 5.1 Second Step IGC01S

After the equilibration of the Capture Select CH1-XL (Thermo Fisher, Cat.Nr.: 2943452050, ID: 1.0 cm h=3.8 cm A=0.79 cm² V=3.0 ml) with IQEQ buffer the MAB Select PrismA flow through (IGC02) was applied onto the column with a contact time of approx. 10 min.

After the load, a first wash was applied with 5 CV of buffer IQEQ to remove unbound proteins from the resin (added to flowthrough). A SD_VI on column treatment was carried out by applying IQEQ_VI buffer which contains the detergent composition of 10.55 g Triton X-100, 3.21 g Polysorbate 80 and 2.91 g TnBP in 1 kg of IQEQ buffer. The SD_VI on column takes place by contacting the bound proteins for a minimum of 60 minutes with IQEQ_VI buffer with a flow rate of 0.3 ml/min within 10 CV (100 min).

Wash 2 was carried out with IQEQ buffer for further depletion of impurities and to wash out the SD_VI reagents in 20 CV's and a flow rate of 0.8 ml/min.

The elution was performed by applying 10 CV's of E2 buffer (100 mM Glycine, pH 2.7).

After a re-equilibration of 10 CV of IQEQ buffer the column were regenerated and stored.

Regeneration procedure and storage are described under Regeneration Procedure.

### 5.2 Column

Capture Select CH1XL
Thermo Fisher, Cat.Nr.: 2943452050
Lot.Nr.: 170426-01
ID: 10 cm h=3.8 cm A=0.79 cm² V=3.0 ml

TABLE 15
Chromatography Scheme

| | | Inlet | | Flowrate | Outlet | |
|---|---|---|---|---|---|---|
| Chromatography Step | Buffer/Sample | Valve | CV | (ml/min) | valve | Fraction |
| Equilibration | IQEQ | A1 | — | 0.30 | — | Waste |
| Resin activation | 100 mM Glycine, pH 2.7 | B1 | 5 | 0.30 | — | Waste |
| Equilibration | IQEQ | A1 | 10 | 0.30 | — | Waste |
| Sample application | — | S1 | 45-50 mL | 0.30 | 1 | FT |
| WASH 1 Re-equilibration | IQEQ | A1 | 5 | 0.30 | 1 | FT |
| SD_VI ON Column | IQEQ_VI | A2 | 10 | 0.30 | 2 | WSD |
| WASH 2 | IQEQ | A1 | 20 | 0.80 | 3 | W2 |
| Elution1 | E2 | B1 | 10 | 0.30 | 4 | E1 |
| Reconditioning | IQEQ | A1 | 10 | 1.00 | — | Waste |

a. Chromatogram IGC01S

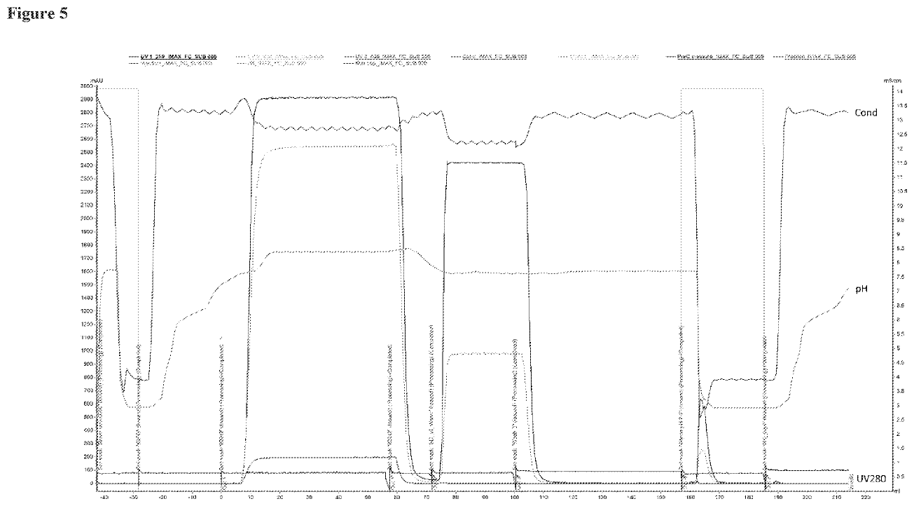

See, FIG. 5.

### 5.3 Results of Run IGC02

TABLE 16
IGC02 IgG1, IGg2, IgG3 and IgG4 in the
“PrismA Flowthrough(FT)”

| | IgG1 | IgG2 | IgG3 | IgG4 |
|---|---|---|---|---|
| LOAD [mg] | 761.5 | 338.2 | 51.66 | 65.77 |
| FT [mg] | 34.43 | x^{a} | 36.36 | x^{a} |
| % in FT ² | 4.5% | x^{a} | 70.4% | x^{a} |

¹ Ratio based wherein IgG3 is 1 part/Ratio are given as the x-fold part of IgG3
² Percent of IgG subclasses found in the PrismA Flow through
Only IgG3 is found in significant amounts in the flow through of Mab Select PrismA.
x^{a} Values below detection limit

TABLE 17
IgG1 and IgG3 on Capture Select CH1_XL

| | IgG1 | | IgG3 | |
|---|---|---|---|---|
| | mg | IgG1 % | mg | IgG3 % |
| LOAD [mg] | 5.8 | 100% | 6.2 | 100% |
| FT [mg] | x^{a} | x^{a} | x^{a} | x^{a} |
| Eluate | 3.0 | 51.6% | 3.6 | 57.6% |

x^{a} Values below detection limit
{Note:
this column captures essentially all IgG1 and IgG3}

### Example 6

### 6.1 Variant IGC04

a. Buffer Compositions Used in Example 6

TABLE 18
Buffer Compositions

| Buffer | Buffer ID | Buffer composition | pH | Name |
|---|---|---|---|---|
| 1. | Equilibration/ | 10 mM NaAcetate, | 7.2 | IQEQ |
| | Dilution buffer | 10 mM TrisHCl, | | |
| | | 120 mM NaCl | | |
| 2. | WASH/SD_VI | 10 mM NaAcetate, | 7.2 | IQEQ_VI |
| | On Column | 10 mM TrisHCl, | | |
| | buffer | 120 mM NaCl + | | |
| | | SD_VI_MIX Triton X | | |
| | | 10.55 g/Polysorbate 80 | | |
| | | 3.21 g/TnBP 2.91 g | | |
| | | per kg buffer | | |
| 3. | Wash 2 High | 100 mM NaAcetate, | 6.0 | W2 |
| | Salt | 500 mM NaCl | | |
| | | 0.05% Polysorbate | | |
| 4. | Elution buffer 1 | 100 mM Glycine | 3.2 | E1 |
| 5. | Elution buffer 2 | 100 mM Glycine | 2.7 | E2 |
| 6. | Adjusting buffer | 2M NaAcetate | 6.1 | Adj |

### 6.2 IGC04

Cryo-supernatant from the Cohn was the starting material for the purification process without any further treatment prior the experiment. The experiment was carried out at +2 to +8° C.

After the equilibration of an array of MAB-Select PrismA (GE Healthcare, Cat.Nr.: 17-5498-02, ID: 3.2 cm h=7.5 cm A=8.04 cm² V=60.3 ml) and Capture Select CH1-XL Thermo Fisher, Cat.Nr.: 2943452050) ID: 2.2 cm h=3.6 cm A=3.81 cm² V=13.7 ml in series (flowthrough of the first column directly loaded onto the second column) the cryo-supernatant was applied onto the columns with a contact time of approx. 6 min for the CH1-XL column [2.3 ml/min].

After the load, a first wash was applied with 2 CV of buffer IQEQ to remove unbound proteins from the resin. A SD_VI on column treatment was carried out by applying IQEQ_VI buffer which contains the detergent composition of 10.55 g Triton X-100, 3.21 g Polysorbate 80 and 2.91 g TnBP in 1 kg of IQEQ buffer (mixing the components of the detergent composition for about 15 minutes; subsequent addition to the IQEQ buffer under mixing). The SD_VI on column is run by contacting the bound proteins for a minimum of 60 minutes with IQEQ_VI buffer with a flow rate of 4 ml/min within 4 CV. For the SD_VI step: 16.6 g/Kg SD_VI_MIX is added to 10 mM NaAcetate, 10 mM TrisHCl, 120 mM NaCl, pH 7.2. The SD_VI_MIX was prepared by mixing the detergents/solvents in a ratio of 10.55 g Triton X100/3.21 g Polysorbate 80/2.91 g TnPB (Tri-n-buthylphosphate) for a minimum of 15 min and add it to the buffer under mixing.

Wash 2 was carried out with W2 buffer for further depletion of impurities and to wash out the SD_VI reagents in 5 CV's and a flow rate of 10 ml/min. The 3^{rd} and final wash step was carried out by applying 6 CV's of IQEQ buffer with a flow rate of 10 ml/min.

The elution was performed by applying 5 CV's of E1 buffer (100 mM Glycine, pH 3.2).

The 1^{st} step of a cleaning procedure (Strip) was carried out with E2 buffer (100 mM Glycine, pH 2.7).

After a re-equilibration of 4 CV of IQEQ buffer the columns were regenerated and stored separately.

CV is the total volume of both columns (CH1-XL and Capture Select CH1-XL). Regeneration procedure and storage are described under Regeneration Procedure.

### Example 7

The eluate from Example 6 (IGC04) was processed as described below. This example is described as Example 6-Variant IGC04.

### 7.1 Tandem Column Array

a. Column 1
MAB Select Prism A
GE Healthcare, Cat.Nr.: 17-5498-02
Lot.Nr.: 10264190
ID: 3.2 cm h=7.5 cm A=8.04 cm² V=60.3 ml (Target)
   b. Column 2
Capture Select CH1XL
Thermo Fisher, Cat.Nr.: 2943452050
Lot.Nr.: 170426-01
ID: 2.2 cm h=3.6 cm A=3.81 cm² V=13.7 ml
Column volume 1+2:74.0 ml Programmed: 74.506 (Column1+2+connection—defined as 1 CV)

TABLE 19
Chromatography Scheme

| | | Inlet | | Flowrate | Outlet | |
|---|---|---|---|---|---|---|
| Chromatography Step | Buffer/Sample | Valve | CV | (ml/min) | valve | Fraction |
| Equilibration | IQEQ | A1 | 2 | 10 | — | Waste |
| Resin activation | 100 mM Glycine, pH | B1 | 3 | 10 | — | Waste |
| | 2.7 | | | | | |
| Equilibration | IQEQ | A1 | 5 | 10 | — | Waste |
| Sample application | Cryo supernatant | S1 | mL | 2.5* | 1 | FT |
| WASH 1 Re- | IQEQ | A1 | 2 | 2.5* | 1 | FT |
| equilibration | | | | | | |
| SD_VI ON Column | IQEQ_VI | A2 | 4 | 4.0 | 2 | WSD |
| WASH 2 | W2 | A3 | 5 | 10 | 3 | W2 |
| WASH 3 | IQEQ | A1 | 6 | 10 | 4 | W3 |
| Elution1 | E1 | B1 | 5 | 5 | 5 | E1 |
| Elution2 | E2 | B2 | 5 | 5 | 6 | STRIP/E2 |
| Reconditioning | IQEQ | A1 | 4 | 10 | — | Waste |

Column volume 1 + 2: 74.0 ml Programmed: 74.506 (Column1 + 2 + connection − defined as 1 CV)
*Contact time approx. 6 min on column
Fraction WSD was sampled and analysed in detail. Fractions W2 and W3 were sampled and analysed in SDS PAGE silver stain.

c. Chromatogram IGC04

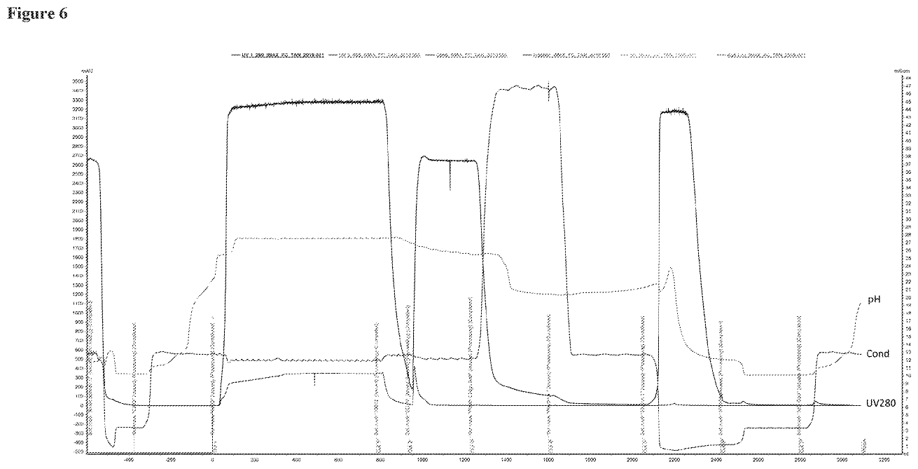

See, FIG. 6.

### 7.2 Results and Data for Example 7

TABLE 20
Concentration of IgG as Determined
by Immunological Analysis (ELISA)

| Fraction | LOAD | FT | ELUATE | STRIP |
|---|---|---|---|---|
| Concentration | 4.92 | 0.23 | 8.70 | 0.03 |
| IgG | mg/ml | mg/ml | mg/ml | mg/ml |

TABLE 21
Yield IgG Subclass Distribution Load vs Eluate

| | | | | Step |
|---|---|---|---|---|
| SUBCLASS | RANGE | LOAD | ELUATE | yield |
| IgG 1 | 50-70% | 62.3% | 65.8% | 87.1% |
| IgG 2 | 26.7-37.3% | 28.1% | 28.2% | 82.6% |
| IgG 3 | >2.5% | 6.4% | 2.6% | 34.0% |
| IgG 4 | >2.5% | 3.2% | 3.4% | 86.3% |
| IgA | n.a | 100% | 12.0% | 12.0% |
| IgG | n.a | 100% | 92.9% | 92.9% |
| IGM | n.a | 100% | 22.5% | 22.5% |

n.a = not applicable

### Product/Impurity Profile

LC-MS Data was generated regarding the protein profile of IMAX_FC_CRS_04 WSD (the wash buffer containing the SD_VI reagents) and the eluate IMAX_FC_CRS_04 (eluate of two column in series: MAB Select Prism A and Captur Select CH1_XL) which represents the intermediate product fraction after the affinity purification. The impurity profile of the SD_VI on column viral inactivation wash fraction, and the product/impurity profile of the eluate were determined by RP-HPLC-ESI-MS/MS. The samples were precipitated with TCA, reduced, alkylated, digested with Trypsin and subsequently analyzed with an Thermo UltiMate Cap-HPLC coupled to an Eclipse Orbitrap (RP-HPLC-ESI-MSMS analysis). The data was analyzed with “Proteome Discoverer” software. The results consist of protein composition and relative protein abundance for each sample (label free quantification).

### Results

The results are set out in Table 22 and Table 23. All different detected variants of immunoglobulins were merged into one protein entry “Immunoglobulin” and all respective area % values were summarized. Accession numbers are from UniProt KB. An area % value of 0.00% means that the protein could be detected and identified but the relative amount is below the threshold of 0.01%

### CONCLUSION

The fraction IMAX_FC_CRS_04 WSD (“the wash fraction”) contains impurities which are depleted during the SD_VI on column. The “wash fraction” sample has a protein composition of 66.96% Immunoglobulins and 33.04% other proteins. 150 different proteins could be detected and identified. (Table 22). There was essentially 0% Serum amyloid A-2 protein, Prenylcysteine oxidase 1, Leucine-rich alpha-2-glycoprotein, Coagulation factor XI, Proteoglycan 4, Carboxypeptidase B2, Sex hormone-binding globulin, Adiponectin, Hyaluronan-binding protein 2, Neutrophil defensin 1, Ras-related protein Rap-1b, Transforming growth factor-beta-induced protein ig-h3, Integrin beta-3, Properdin, or Carboxypeptidase N catalytic chain in the WSD.

The “eluate” sample has a protein composition of 99.57% Immunoglobulins and 0.43% other proteins. 26 different proteins could be detected and identified. (Table 23). The eluate represents a highly pure human IgG pool (>99%) which is superior compared to current commercially available IgG products. The affinity eluate contained essentially 0% of Alpha-1-acid glycoprotein 2, Transthyretin, Apolipoprotein B-100, C4b-binding protein alpha chain, Fibronectin, Histidine-rich glycoprotein, Complement C5, or Pregnancy zone protein.

TABLE 22
Impurity Profile of the WSD (Wash Fraction)

| Accession | Description | Abundance | Area % |
|---|---|---|---|
| | Immunoglobulins | 2.91E+10 | 66.96 |
| P02768 | Albumin | 3.69E+09 | 8.49 |
| P02647 | Apolipoprotein A-I | 1.36E+09 | 3.14 |
| P01024 | Complement C3 | 9.86E+08 | 2.27 |
| P00738 | Haptoglobin | 8.37E+08 | 1.93 |
| P02679 | Fibrinogen gamma chain | 4.54E+08 | 1.04 |
| P0C0L5 | Complement C4-B | 3.89E+08 | 0.89 |
| P02675 | Fibrinogen beta chain | 3.48E+08 | 0.80 |
| P02671 | Fibrinogen alpha chain | 3.41E+08 | 0.78 |
| P09871 | Complement C1s subcomponent | 3.27E+08 | 0.75 |
| P00736 | Complement C1r subcomponent | 3.20E+08 | 0.74 |
| P04114 | Apolipoprotein B-100 | 2.91E+08 | 0.67 |
| P01023 | Alpha-2-macroglobulin | 2.49E+08 | 0.57 |
| P02656 | Apolipoprotein C-III | 2.26E+08 | 0.52 |
| P02790 | Hemopexin | 2.17E+08 | 0.50 |
| P02652 | Apolipoprotein A-II | 2.10E+08 | 0.48 |
| P02787 | Serotransferrin | 2.08E+08 | 0.48 |
| Q03591 | Complement factor H-related | 2.06E+08 | 0.48 |
| | protein 1 | | |
| P08603 | Complement factor H | 1.86E+08 | 0.43 |
| P01009 | Alpha-1-antitrypsin | 1.55E+08 | 0.36 |
| P02766 | Transthyretin | 1.37E+08 | 0.31 |
| P10909 | Clusterin | 1.28E+08 | 0.30 |
| O14791 | Apolipoprotein L1 | 1.27E+08 | 0.29 |
| P08519 | Apolipoprotein(a) | 1.13E+08 | 0.26 |
| P04004 | Vitronectin | 1.11E+08 | 0.25 |
| Q4G0P3 | Hydrocephalus-inducing | 1.03E+08 | 0.24 |
| | protein homolog | | |
| P19823 | Inter-alpha-trypsin inhibitor | 9.29E+07 | 0.21 |
| | heavy chain H2 | | |
| Q15485 | Ficolin-2 | 9.14E+07 | 0.21 |
| P04003 | C4b-binding protein alpha chain | 8.80E+07 | 0.20 |
| P00747 | Plasminogen | 8.24E+07 | 0.19 |
| P01031 | Complement C5 | 7.34E+07 | 0.17 |
| P02649 | Apolipoprotein E | 6.95E+07 | 0.16 |
| P02760 | Protein AMBP | 6.94E+07 | 0.16 |
| P05155 | Plasma protease C1 inhibitor | 6.89E+07 | 0.16 |
| P02743 | Serum amyloid P-component | 6.68E+07 | 0.15 |
| P01042 | Kininogen-1 | 6.51E+07 | 0.15 |
| P19827 | Inter-alpha-trypsin inhibitor | 6.37E+07 | 0.15 |
| | heavy chain H1 | | |
| P02763 | Alpha-1-acid glycoprotein 1 | 6.28E+07 | 0.14 |
| P00450 | Ceruloplasmin | 6.20E+07 | 0.14 |
| P05090 | Apolipoprotein D | 6.15E+07 | 0.14 |
| Q14624 | Inter-alpha-trypsin inhibitor | 5.89E+07 | 0.14 |
| | heavy chain H4 | | |
| O43866 | CD5 antigen-like | 5.73E+07 | 0.13 |
| P00739 | Haptoglobin-related protein | 4.62E+07 | 0.11 |
| P05156 | Complement factor I | 4.42E+07 | 0.10 |
| P02765 | Alpha-2-HS-glycoprotein | 4.42E+07 | 0.10 |
| P07358 | Complement component C8 | 4.38E+07 | 0.10 |
| | beta chain | | |
| P01011 | Alpha-1-antichymotrypsin | 4.34E+07 | 0.10 |
| P02655 | Apolipoprotein C-II | 4.21E+07 | 0.10 |
| P07360 | Complement component C8 | 4.19E+07 | 0.10 |
| | gamma chain | | |
| P04196 | Histidine-rich glycoprotein | 4.13E+07 | 0.10 |
| P27169 | Serum paraoxonase/arylesterase 1 | 4.09E+07 | 0.09 |
| P07357 | Complement component C8 | 3.99E+07 | 0.09 |
| | alpha chain | | |
| P02774 | Vitamin D-binding protein | 3.86E+07 | 0.09 |
| P02749 | Beta-2-glycoprotein 1 | 3.52E+07 | 0.08 |
| Q15648 | Mediator of RNA polymerase II | 3.34E+07 | 0.08 |
| | transcription subunit 1 | | |
| P19652 | Alpha-1-acid glycoprotein 2 | 3.33E+07 | 0.08 |
| P0C0L4 | Complement C4-A | 3.24E+07 | 0.07 |
| P36980 | Complement factor H-related | 3.16E+07 | 0.07 |
| | protein 2 | | |
| P35542 | Serum amyloid A-4 protein | 2.93E+07 | 0.07 |
| P00734 | Prothrombin | 2.88E+07 | 0.07 |
| P02654 | Apolipoprotein C-I | 2.79E+07 | 0.06 |
| P02751 | Fibronectin | 2.69E+07 | 0.06 |
| P06396 | Gelsolin | 2.33E+07 | 0.05 |
| P22792 | Carboxypeptidase N subunit 2 | 2.29E+07 | 0.05 |
| P06727 | Apolipoprotein A-IV | 2.27E+07 | 0.05 |
| P23142 | Fibulin-1 | 2.21E+07 | 0.05 |
| P61626 | Lysozyme C | 2.19E+07 | 0.05 |
| P04217 | Alpha-1B-glycoprotein | 2.19E+07 | 0.05 |
| P68871 | Hemoglobin subunit beta | 2.10E+07 | 0.05 |
| Q15166 | Serum paraoxonase/lactonase 3 | 2.06E+07 | 0.05 |
| P02753 | Retinol-binding protein 4 | 2.04E+07 | 0.05 |
| P02748 | Complement component C9 | 1.94E+07 | 0.04 |
| O95445 | Apolipoprotein M | 1.94E+07 | 0.04 |
| P11226 | Mannose-binding protein C | 1.93E+07 | 0.04 |
| P01008 | Antithrombin-III | 1.86E+07 | 0.04 |
| P01019 | Angiotensinogen | 1.70E+07 | 0.04 |
| P00751 | Complement factor B | 1.70E+07 | 0.04 |
| O75636 | Ficolin-3 | 1.65E+07 | 0.04 |
| P02746 | Complement C1q subcomponent | 1.59E+07 | 0.04 |
| | subunit B | | |
| P69905 | Hemoglobin subunit alpha | 1.57E+07 | 0.04 |
| P48740 | Mannan-binding lectin serine | 1.56E+07 | 0.04 |
| | protease 1 | | |
| P03952 | Plasma kallikrein | 1.48E+07 | 0.03 |
| P02747 | Complement C1q subcomponent | 1.48E+07 | 0.03 |
| | subunit C | | |
| P07225 | Vitamin K-dependent protein S | 1.41E+07 | 0.03 |
| Q15113 | Procollagen C-endopeptidase | 1.34E+07 | 0.03 |
| | enhancer 1 | | |
| Q02985 | Complement factor H-related | 1.25E+07 | 0.03 |
| | protein 3 | | |
| P08697 | Alpha-2-antiplasmin | 1.09E+07 | 0.03 |
| P20851 | C4b-binding protein beta chain | 1.01E+07 | 0.02 |
| Q6ZRK6 | Coiled-coil domain-containing | 9.44E+06 | 0.02 |
| | protein 73 | | |
| Q13790 | Apolipoprotein F | 9.42E+06 | 0.02 |
| P55056 | Apolipoprotein C-IV | 9.09E+06 | 0.02 |
| Q06033 | Inter-alpha-trypsin inhibitor | 8.96E+06 | 0.02 |
| | heavy chain H3 | | |
| P06681 | Complement C2 | 8.91E+06 | 0.02 |
| O00187 | Mannan-binding lectin serine | 8.89E+06 | 0.02 |
| | protease 2 | | |
| P34096 | Ribonuclease 4 | 8.83E+06 | 0.02 |
| P20742 | Pregnancy zone protein | 8.77E+06 | 0.02 |
| P02745 | Complement C1q subcomponent | 8.73E+06 | 0.02 |
| | subunit A | | |
| P43652 | Afamin | 8.46E+06 | 0.02 |
| P29622 | Kallistatin | 8.43E+06 | 0.02 |
| Q16610 | Extracellular matrix protein 1 | 8.32E+06 | 0.02 |
| P18428 | Lipopolysaccharide-binding | 8.06E+06 | 0.02 |
| | protein | | |
| P05160 | Coagulation factor XIII B chain | 8.04E+06 | 0.02 |
| P60709 | Actin, cytoplasmic 1 | 7.65E+06 | 0.02 |
| P49908 | Selenoprotein P | 7.37E+06 | 0.02 |
| O00391 | Sulfhydryl oxidase 1 | 7.32E+06 | 0.02 |
| P00488 | Coagulation factor XIII A chain | 7.30E+06 | 0.02 |
| P05546 | Heparin cofactor 2 | 7.04E+06 | 0.02 |
| Q08380 | Galectin-3-binding protein | 6.94E+06 | 0.02 |
| P25311 | Zinc-alpha-2-glycoprotein | 6.83E+06 | 0.02 |
| Q92496 | Complement factor H-related | 6.34E+06 | 0.01 |
| | protein 4 | | |
| Q96PD5 | N-acetylmuramoyl-L-alanine | 6.24E+06 | 0.01 |
| | amidase | | |
| Q12805 | EGF-containing fibulin-like | 6.06E+06 | 0.01 |
| | extracellular matrix protein 1 | | |
| P55058 | Phospholipid transfer protein | 5.63E+06 | 0.01 |
| P13671 | Complement component C6 | 5.27E+06 | 0.01 |
| Q5TF21 | Protein SOGA3 | 5.06E+06 | 0.01 |
| P00748 | Coagulation factor XII | 5.04E+06 | 0.01 |
| P61769 | Beta-2-microglobulin | 4.85E+06 | 0.01 |
| P63267 | Actin, gamma-enteric smooth | 4.65E+06 | 0.01 |
| | muscle | | |
| P51884 | Lumican | 4.00E+06 | 0.01 |
| P01833 | Polymeric immunoglobulin | 3.98E+06 | 0.01 |
| | receptor | | |
| P35858 | Insulin-like growth factor-binding | 3.85E+06 | 0.01 |
| | protein complex acid labile subunit | | |
| O75015 | Low affinity immunoglobulin | 3.60E+06 | 0.01 |
| | gamma Fc region receptor III-B | | |
| P36955 | Pigment epithelium-derived | 3.58E+06 | 0.01 |
| | factor | | |
| P10643 | Complement component C7 | 3.43E+06 | 0.01 |
| P22352 | Glutathione peroxidase 3 | 3.26E+06 | 0.01 |
| P04070 | Vitamin K-dependent protein C | 3.24E+06 | 0.01 |
| P08514 | Integrin alpha-IIb | 3.08E+06 | 0.01 |
| Q96KN2 | Beta-Ala-His dipeptidase | 3.03E+06 | 0.01 |
| Q9NZP8 | Complement C1r | 2.91E+06 | 0.01 |
| | subcomponent-like protein | | |
| Q9BXR6 | Complement factor H-related | 2.63E+06 | 0.01 |
| | protein 5 | | |
| O75882 | Attractin | 2.55E+06 | 0.01 |
| P11597 | Cholesteryl ester transfer protein | 2.53E+06 | 0.01 |
| P02730 | Band 3 anion transport protein | 2.48E+06 | 0.01 |
| P12259 | Coagulation factor V | 2.48E+06 | 0.01 |
| P03950 | Angiogenin | 2.40E+06 | 0.01 |
| | SUM: | 4.35E+10 | 100.00 |

TABLE 23
Product/Impurity Profile of the Affinity Eluate E1

| Accession | Description | Abundance | Area % |
|---|---|---|---|
| | Immunoglobulins | 1.81E+11 | 99.57 |
| P0C0L5 | Complement C4-B | 1.50E+08 | 0.08 |
| P01024 | Complement C3 | 8.84E+07 | 0.05 |
| P02768 | Albumin | 8.25E+07 | 0.05 |
| P02679 | Fibrinogen gamma chain | 7.88E+07 | 0.04 |
| P02675 | Fibrinogen beta chain | 7.28E+07 | 0.04 |
| P02671 | Fibrinogen alpha chain | 6.03E+07 | 0.03 |
| O43866 | CD5 antigen-like | 4.91E+07 | 0.03 |
| P0C0L4 | Complement C4-A | 2.23E+07 | 0.01 |
| P00739 | Haptoglobin-related protein | 2.00E+07 | 0.01 |
| P02745 | Complement C1q | 1.76E+07 | 0.01 |
| | subcomponent subunit A | | |
| P01833 | Polymeric immunoglobulin | 1.72E+07 | 0.01 |
| | receptor | | |
| P01009 | Alpha-1-antitrypsin | 1.67E+07 | 0.01 |
| P02746 | Complement C1q | 1.60E+07 | 0.01 |
| | subcomponent subunit B | | |
| P02747 | Complement C1q | 1.47E+07 | 0.01 |
| | subcomponent subunit C | | |
| P02760 | Protein AMBP | 1.38E+07 | 0.01 |
| P01023 | Alpha-2-macroglobulin | 1.25E+07 | 0.01 |
| P10909 | Clusterin | 1.00E+07 | 0.01 |
| P00738 | Haptoglobin | 9.51E+06 | 0.01 |
| | Sum: | 1.81E+11 | 100.00 |

TABLE 24
Impurity Profile of the Affinity Eluate
(LC-MS Run IMAX_FC_CRS_04)

| | | |
|---|---|---|
| IMPURITIES | ELUATE | LOAD |

| | | | |
|---|---|---|---|
| Albumin | <0.359 | g/L | x. |
| Factor XI^{#} | <0.01 | E/ml | x. |
| Alpha2-Macro | <0.0478 | g/L | x. |
| Transferrin | <0.0897 | g/L | x. |
| PL1 Activity | <10 | nm/ml min | x. |
| Fibrinogen | 29.9 | μg/ml | x. |
| Amidolyt S2222 | <5 | nm/ml min | 7.57 |
| Amidolyt S2251 | <5 | nm/ml min | 5.91 |
| Amidolyt S2288 | <5 | nm/ml min | VK > 30% |
| Amidolyt S2302 | <5 | nm/ml min | 6.86 |
| C3 ELISA | 18.0 | μg | x. |

^{#}Determined by ELISA

a. SDS PAGE Silver Stain Profile

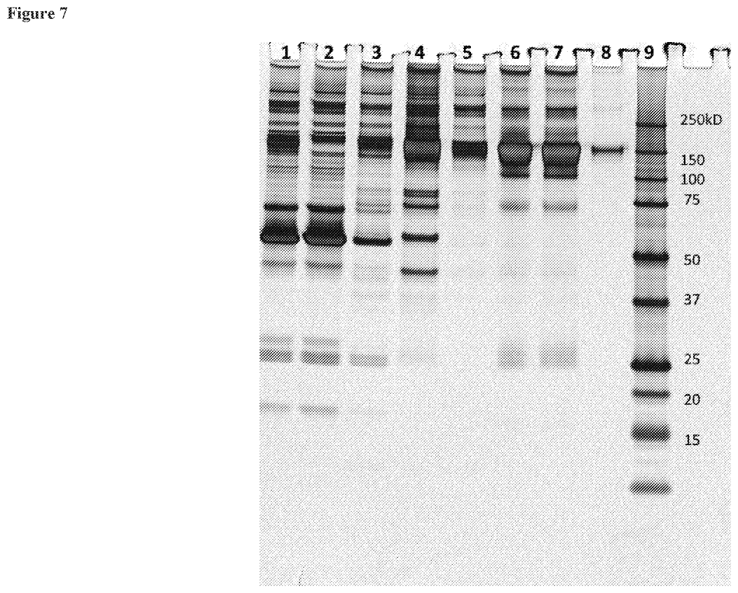

See FIG. 7.

### Example 8

8.1
a. Buffer Compositions used in Example 8

TABLE 25
Buffer Compositions

| Buffer | Buffer ID | Buffer composition | pH | Name |
|---|---|---|---|---|
| 1. | Equilibration/ | 10 mM NaAcetate, | 7.2 | IQEQ |
| | Dilution buffer | 10 mM TrisHCl, | | |
| | | 120 mM NaCl | | |
| 2. | WASH/SD_VI | 10 mM NaAcetate, | 7.2 | IQEQ_VI |
| | On Column | 10 mM TrisHCl, | | |
| | buffer | 120 mM NaCl + | | |
| | | SD_VI_MIX Triton X | | |
| | | 10.55 g/Polysorbate 80 | | |
| | | 3.21 g/TnBP 2.91 gper | | |
| | | kg buffer | | |
| 3. | Wash 2 High | 100 mM NaAcetate, | 6.0 | W2 |
| | Salt | 500 mM NaCl | | |
| | | 0.05% Polysorbate | | |
| 4. | Elution buffer 1 | 100 mM Glycine | 3.2 | E1 |
| 5. | Elution buffer 2 | 100 mM Glycine | 2.7 | E2 |
| 6. | Adjusting buffer | 2M NaAcetate | 6.1 | Adj |

### 8.1. Run

Cryo-supernatant from the Cohn-fractionation process was used as starting material for the purification process without any further treatment prior the experiment. After the equilibration of an array of 3 columns of MAB-Select PrismA (GE Healthcare, Cat. Nr.: 17-5498-02, ID: 3.2 cm h=7.5 cm A=8.04 cm² V=60.3 ml) and Capture Select CH1-XL Thermo Fisher, Cat.Nr.: 2943452050, ID: 2.2 cm h=3.6 cm A=3.81 cm² V=13.7 ml) and Capture Select IgG3 (Thermo Fisher, Cat.Nr.: 191304010, ID: 2.2 cm h=3.1 cm A=3.81 cm² V=11.8 ml) in series the cryo-supernatant was applied onto the columns with a contact time of approx. 6 min for the CH1-XL column [2.3 ml/min].

After the load, a first wash was applied with 2 CV of buffer IQEQ to remove unbound proteins from the resin. A SD_VI on column treatment was carried out by applying IQEQ_VI buffer which contains the detergent composition of 10.55 g Triton X-100, 3.21 g Polysorbate 80 and 2.91 g TnBP in 1 kg of IQEQ buffer. The SD_VI on column takes place by contacting the bound proteins for a minimum of 60 minutes with IQEQ_VI buffer with a flow rate of 4 ml/min within 4 CV.

Wash 2 was carried out with W2 buffer for further depletion of impurities and to wash out the SD_VI reagents in 5CV's and a flow rate of 10 ml/min. The 3rd and final wash step was carried out by applying 6 CV's of IQEQ buffer with a flow rate of 10 ml/min.

The elution was performed by applying 5 CV's of E1 buffer (100 mM Glycine, pH 3.2). The 1st step of a cleaning procedure (Strip) was carried out with E2 buffer (100 mM Glycine, pH 2.7). After a re-equilibration of 4 CV of IQEQ buffer the columns were regenerated and stored separately.

### 8.2 Tridem Column Array:

a. Column 1
MAB Select Prism A
GE Healthcare, Cat.Nr.: 17-5498-02
Lot.Nr.: 10264190
ID: 3.2 cm h=7.5 cm A=8.04 cm² V=60.3 ml
   b. Column 2
Capture Select CH1XL
Thermo Fisher, Cat.Nr.: 2943452050
Lot.Nr.: 170426-01
ID: 2.2 cm h=3.6 cm A=3.81 cm² V=13.7 ml
   c. Column 3
Thermo Fisher, Cat.Nr.: 191304010
Lot.Nr.: 151012-01
ID: 2.2 cm h=3.1 cm A=3.81 cm² V=11.8 ml
Column volume 1+2+3:85.8 ml Programmed: 86.4 (Column1+2+3+connection)

### 8.3 Chromatogram

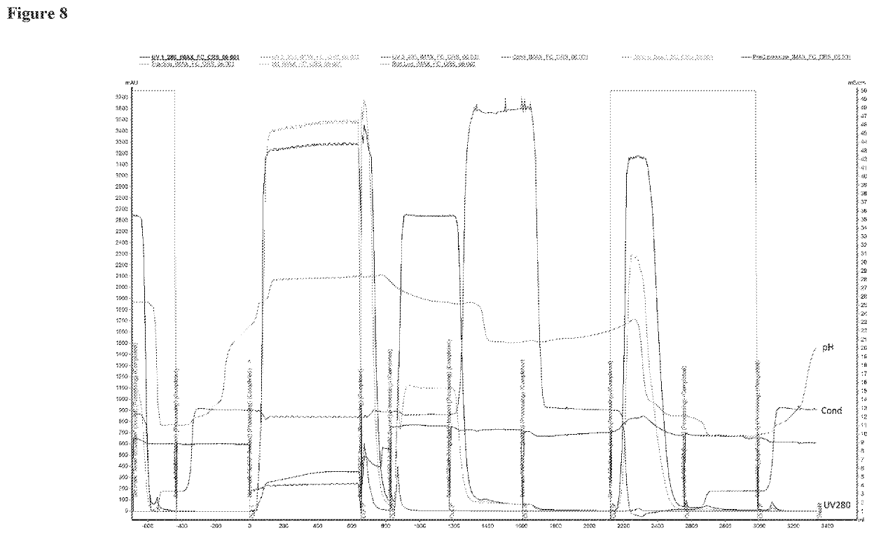

See FIG. 8.

### 8.4 Results and Data of Run

a. Yield IgG Subclass Distribution Load vs Eluate

The Tridem array provides a highly efficient method to purify a Cohn fraction to an IgG-pattern comparable to the IgG subclass distribution of plasma. The ratio of the IgG subclasses is almost identical to the pattern in commercial IVIG products. Only the amount of IgG4 is higher. The SDS-PAGE Silver stain confirms a high purity of the affinity eluate.

TABLE 26
Yield IgG Subclass Distribution Load vs Eluate

| | ACCEPTANCE | | | Step |
|---|---|---|---|---|
| SUBCLASS | RANGE* | LOAD | ELUATE | yield |
| IgG 1 | 50-70% | 56.8% | 58.3% | 90.9% |
| IgG 2 | 26.7-37.3% | 31.6% | 32.2% | 89.7% |
| IgG 3 | >2.5% | 7.7% | 5.6% | 64.1% |
| IgG 4 | >2.5% | 3.9% | 3.9% | 87.5% |
| IgA | n.a | 100% | 12.8% | 12.8% |
| IgG | n.a | 100% | 91.5% | 91.5% |
| IGM | n.a | 100% | 30.3% | 30.3% |

*Reference, Human serum

b. Impurity Profile of the Affinity Eluate

TABLE 27
Impurity Profile of the Affinity Eluate (LC-MS
Run IMAX_FC_CRS_06)*

| | | |
|---|---|---|
| IMPURITIES | ELUATE | LOAD |

| Albumin | <0.335 | g/L | x | |
|---|---|---|---|---|
| Factor XI | <0.01 | E/ml | 0.93 | E/ml |
| Alpha2-Macro | <0.185 | g/L | x | |
| Transferrin | <0.0884 | g/L | x | |
| PL1 Activity | <10 | nm | x | |
| Fibrinogen | 13.0 | μg/ml | x | |
| Amidolyt S2222 | <5 | nm/ml min | 6.51 | nm/ml min |
| Amidolyt S2251 | <5 | nm/ml min | 5.56 | nm/ml min |
| Amidolyt S2288 | <5 | nm/ml min | 10.9 | nm/ml min |
| Amidolyt S2302 | <5 | nm/ml min | 7.45 | nm/ml min |

| | | | |
|---|---|---|---|
| C3 ELISA | 4.8 | μg/ml | x |

*Three immunoaffinity columns in series

b. SDS PAGE Silver Stain Profile

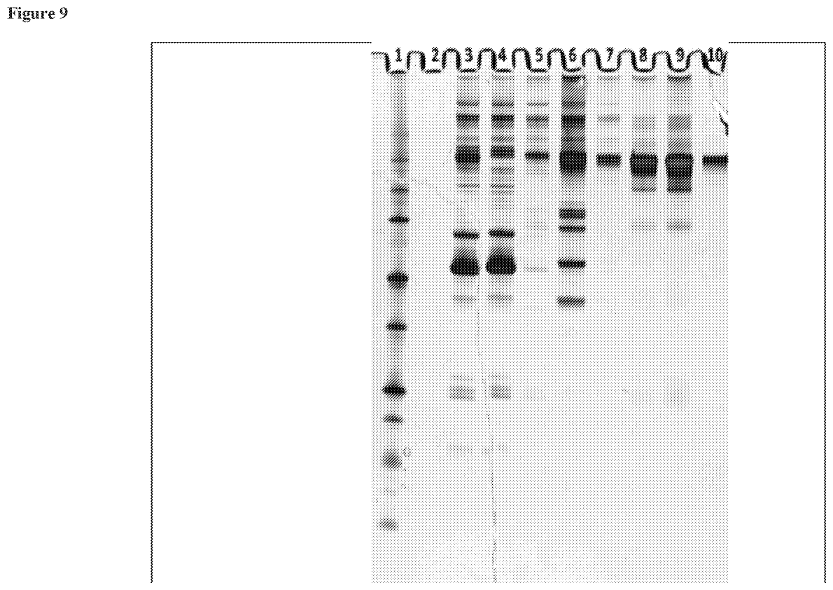

See FIG. 9.

A broad pattern of IgG subclasses was obtained from IgG-containing start material (cryo supernatant, Gammagard liquid) by using a combination of two and/or three columns containing complementary immunoglobulin binding resins in series. The purity of the obtained intermediate (eluate) is surprisingly high and can be compared with affinity purification steps used in monoclonal antibody purification.

### Example 9

### 9.1 Equipment

For these experiments an ÄKTA was used. The system was equipped with probes that enabled online monitoring of UV absorption (2 mm flow cell), conductivity, pressure, temperature and pH with electronic recording. The chromatography unit was operated with the system software Unicorn 7.3.

TABLE 28
Chromatography Skid

| | |
|---|---|
| System | ÄKTA Explorer 100 |
| Serial No. | 1867216 |
| Tubing | 1 mm Peek and 1.25 mm (appr.) PTFE |
| Software | Unicorn 7.3 |
| Temperature [° C.] | Cold temperature +2 to +8° C. |
| Location | I131/BT-D/3/4-39 |

### 9.2 Regeneration Procedure

a. Capto Select PRISM A was Generally Regenerated with 1 M NaOH by Applying 2 CV with a Total Time of 30 Minutes.

TABLE 29
Regeneration Sequence post STRIP PRISM
A (100 mM Glycine pH 2.7) CV: 60 ml

| | | Inlet | | Flowrate | Outlet | |
|---|---|---|---|---|---|---|
| Chromatography Step | Buffer/Sample | Valve | CV | (ml/min) | valve | Fraction |
| Equilibration | IQEQ | A1 | 2 | 10 | — | Waste |
| Regeneration | 1M NaOH | S1 | 2 | 4 | — | Waste |
| Equilibration | IQEQ | A1 | 5 | 10 | — | Waste |
| Storage | IQEQ_VI | A2 | 5 | 10 | — | Waste |

b. Capture Select CH1-XL was Regenerated with 50% (w/w) Ethylene Glycol, 10% (w/w) Ethanolamine, 0.1% Polysorbate 80, pH 8.5 (PDAM-Buffer) Applying 4 CV with a Total Time of 60 Minutes.

TABLE 30
Regeneration Sequence post STRIP
CH1_XL (100 mM Glycine pH 2.7) CV: 15 ml

| | | Inlet | | Flowrate | Outlet | |
|---|---|---|---|---|---|---|
| Chromatography Step | Buffer/Sample | Valve | CV | (ml/min) | valve | Fraction |
| Equilibration | IQEQ | A1 | 2 | 2 | — | Waste |
| Regeneration | PDAM | S1 | 4 | 1 | — | Waste |
| Equilibration | IQEQ | A1 | 5 | 2 | — | Waste |
| Storage | IQEQ_VI | A2 | 5 | 2 | — | Waste |

Techniques and processes that can be applied together with or in addition to the affinity chromatography production process disclosed herein are included in U.S. Pat. No. 10,125,189 (Method to produce a highly concentrated immunoglobulin preparation for subcutaneous use); U.S. Pat. No. 9,468,675 (Removal of serine proteases by treatment with finely divided silicon dioxide); U.S. Pat. No. 8,993,734 (Method to produce an immunoglobulin preparation with improved yield); U.S. Pat. No. 9,782,477 (Fraction I-IV-1 precipitation of immunoglobins from plasma); US20150133636 (Purification of Biological Molecules); US20170218012 (Integrated Continuous Manufacturing of Therapeutic Protein Drug Substances). All of the foregoing references are incorporated by reference herein in their entirety.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

All references cited herein, including those below and including but not limited to all patents, patent applications, and non-patent literature referenced below or in other portions of the specification, are hereby incorporated by reference herein in their entirety.
1) DOI: 10.5772/36427 Affinity Chromatography for Purification of IgG from Human Plasma, Hofbauer L. et al.
2) U.S. Pat. No. 8,945,895B2 Methods of purifying recombinant ADAMTS13 and other proteins and compositions thereof.
3) WO2019133677 Adeno-associated virus purification methods.

What is claimed is:

1. An affinity chromatographic method of preparing from an IgG-containing starting material an IgG formulation comprising one or more of IgG1, IgG2, IgG3 and IgG4, the method comprising:
(a) contacting the IgG-containing starting material with a first chromatographic medium comprising a first solid support having Protein A bound thereto, forming a Protein A bound fraction of an IgG subclass with Protein A binding affinity and forming a first flow through comprising an IgG subclass not binding to the Protein A;
(b) contacting the first flow through with a first affinity ligand chromatographic medium comprising a second solid support having a first affinity ligand with IgG binding affinity bound thereto, forming a first affinity ligand-bound IgG subclass and a second flow through comprising an IgG subclass not bound to the first affinity ligand-bound IgG subclass;
(c) eluting the Protein A bound fraction from the first chromatographic medium, forming a first eluate and contacting the first eluate with the first affinity ligand chromatographic medium, forming a second affinity ligand-bound IgG subclass, and a second flow through; and
(d) eluting a member selected from the first affinity ligand-bound IgG subclass, the second affinity ligand-bound IgG subclass, and a combination thereof from the first affinity ligand column, forming a second eluate.

2. The method according to claim 1, wherein the first flow through and the second eluate are combined on the first affinity ligand chromatographic medium.

3. The method according to claim 2, further comprising:
(e) contacting the second eluate of (d) with a second affinity ligand chromatographic medium comprising a third solid support having a second affinity ligand with IgG binding affinity bound thereto, forming a third affinity ligand-bound IgG subclass and a second flow through comprising a fourth IgG subclass not bound to the second affinity chromatographic medium; and
(f) eluting a member selected from the second affinity ligand bound IgG subclass, the fourth IgG subclass and a combination thereof from the second affinity ligand chromatographic medium, forming a third eluate.

4. The method according to claim 3, wherein the first flow through and the second eluate are combined on the second affinity ligand chromatographic medium.

5. The method according to claim 1, further comprising:
(g) prior to (b), contacting the first chromatographic medium with a buffer/detergent mixture that does not elute the Protein A bound fraction of an IgG subclass with Protein A binding affinity to from the first chromatographic medium.

6. The method according to claim 3, further comprising:
(h) prior to (f), contacting the second affinity ligand chromatographic medium with a buffer/detergent mixture that does not appreciably elute the second affinity ligand-bound IgG subclass from the second affinity ligand chromatographic medium.

7. The method according to claim 5 or 6, wherein the buffer/detergent mixture comprises acetate, Tris, Triton X, tri(n-butyl)phosphate (TnBP).

8. The method according to claim 7 in which the buffer/detergent mixture comprises:
a buffer component having about 10 mM sodium acetate, about 10 mM TrisHCl, and
about 120 mM NaCl, and a detergent component having about 10.55 g Triton X, 3.21 g Polysorbate 80 and about 2.91 g TnBP per kg of the buffer solution (about 16.6 g of SD_VI reagent to 1 kg buffer).

9. The method according to any one of the preceding claims, wherein IgG in the formulation is at least about 90% (wt/wt) of the protein content of the formulation.

10. The method according to any one of the preceding claims, wherein IgG1 in the formulation is about 40% to about 90% (wt/wt) of the protein content of the formulation.

11. The method according to any one of the preceding claims, wherein IgG1 in the formulation is about 50% to about 70% (wt/wt) of the protein content of the formulation.

12. The method according to any one of the preceding claims, wherein IgG2 in the formulation is about 10% to about 50% (wt/wt) of the protein content of the formulation.

13. The method according to any one of the preceding claims, wherein IgG2 in the formulation is about 27% to about 37% (wt/wt) of the protein content of the formulation.

14. The method according to any one of the preceding claims, wherein IgG3 in the formulation is about 0.1% to about 50% (wt/wt) of the protein content of the formulation.

15. The method according to any one of the preceding claims, wherein in IgG3 the formulation is about 0.5% to about 8% (wt/wt) of the protein content of the formulation.

16. The method according to any one of the preceding claims, wherein IgG4 in the formulation is about 3% to about 70% (wt/wt) of the protein content of the formulation.

17. The method according to any one of the preceding claims, wherein IgG4 in the formulation is about 0.5% to about 8% (wt/wt) of the protein content of the formulation.

18. The method according to any one of the preceding claims, wherein:
(i) IgG1 in the formulation is about 50% to about 70% (wt/wt) of the protein content of the formulation;
(ii) IgG2 in the formulation is about 27% to about 37% (wt/wt) of the protein content of the formulation;
(iii) IgG3 the formulation is about 2.5% to about 8% (wt/wt) of the protein content of the formulation; and
(iv) IgG4 in the formulation is about 3% to about 70% (wt/wt) of the protein content of the formulation.

19. The method according to any one of the preceding claims, wherein albumin in the formulation is in an amount less than about 5 g/L of the formulation.

20. The method according to any one of the preceding claims, wherein Factor XI activity in the formulation is less than about 0.01 E/mL.

21. The method according to any one of the preceding claims, wherein alpha-2-macroglobulin present in the formulation is in an amount less than about 0.2 g/L of the formulation.

22. The method according to any one of the preceding claims, wherein transferrin present in the formulation is in an amount less than about 0.09 g/L of the formulation.

23. The method according to any one of the preceding claims, wherein PL1 activity of the formulation is less than about 10 nm/mL min.

24. The method according to any one of the preceding claims, wherein fibrinogen present in the formulation is in an amount less than about 35 μg/mL of the formulation.

25. The method according to any one of the preceding claims, wherein the IgG-containing starting material is a cryosupernatant.

26. The method according to any one of the preceding claims, wherein a member selected from the first chromatographic medium comprising a first solid support having Protein A, the first affinity ligand chromatographic medium comprising a second solid support having a first affinity ligand with IgG binding affinity bound thereto, and a combination thereof is contained in an individual column.

27. The method according to claim 26, wherein two or more of the columns are in fluidic communication within an array, and wherein two or more of the columns comprise a second chromatographic medium and a second affinity ligand chromatographic medium.

28. The method according to claim 27, wherein the outlet of a first column is in fluidic communication with the inlet of a second column.

29. The method according to claim 28, wherein the outlet of the column having Protein A is in fluidic communication with the inlet of the first affinity ligand chromatographic medium.

30. The method according to claim 27, wherein the outlet of the column containing the second chromatographic medium is in fluidic communication with the inlet of the column containing the second affinity ligand chromatographic medium.

31. The method according to any one of the preceding claims, wherein the IgG solution depleted in a Protein A binding IgG subclass comprises a member selected from IgG1, IgG2, IgG3, IgG4 and a combination thereof.

32. The method according to any one of the preceding claims, wherein the first chromatographic medium has selective affinity for CH1 domain of human IgG antibodies.

33. The method according to any one of the preceding claims, wherein the first chromatographic medium has selective affinity for the CH1 domain of a member selected from IgG1 and IgG3, which is greater than the selective affinity for the CH1 domain of a member selected from IgG2 and IgG4.

34. The method according to any one of the preceding claims, wherein eluting from the first chromatographic medium, the first affinity ligand chromatographic medium, the second affinity ligand chromatographic medium utilizes and eluent comprising glycine.

35. The method according to claim 34, wherein the glycine concentration is from about 50 mM to about 150 mM.

36. An affinity chromatographic method of preparing an IgG formulation enriched in a first IgG subclass with low protein A binding affinity from a plasma-derived IgG solution depleted in a Protein A binding IgG subclass, the method comprising:
(a) contacting the IgG solution depleted in a Protein A binding IgG subclass with a first affinity ligand chromatographic medium comprising a first solid support having a first affinity ligand with IgG binding affinity bound thereto, forming a first bound fraction of the first IgG subclass with low protein A binding affinity and a first flow through; and
(b) eluting the first bound fraction of the first IgG subclass with low protein A binding affinity, forming a first eluent comprising the IgG formulation enriched in the first IgG subclass with low protein A binding.

37. The method according to claim 36, further comprising:
(c) prior to (b), contacting the first bound fraction of the first IgG subclass with low protein A binding affinity with a buffer/detergent solution, and subsequently removing the detergent solution from the first affinity ligand chromatographic medium.

38. The method according to claim 36, further comprising:
(d) following (b), contacting the first eluent with a second chromatographic medium comprising a solid support with an affinity ligand having affinity for a second IgG subclass with low protein A binding affinity bound thereto, forming a first bound fraction of a second IgG subclass with low protein A binding affinity; and
(e) eluting from the second chromatographic medium the first bound fraction of the second IgG subclass with low protein A binding affinity, forming a second eluate.

39. The method according to claim 38, further comprising:
(f) prior to (e), contacting the first bound fraction of the second IgG subclass with low protein A binding affinity with a buffer/detergent solution, and subsequently removing the detergent solution from the first chromatographic medium.

40. The method according to claim 36, further comprising:
(g) prior to (a), contacting a plasma-derived IgG solution with a Protein A chromatographic medium comprising Protein A bound to a solid support, forming a Protein A-bound fraction of an IgG subclass with high affinity for Protein A, and the IgG formulation enriched in a first IgG subclass with low protein A binding affinity.

41. The method according to claim 36, further comprising:
(h) eluting the Protein A-bound fraction of an IgG subclass with high affinity for Protein A from the Protein A chromatographic medium, forming a third eluate.

42. The method of any of claims 36-41, further comprising a step of combining two or more of the first eluate, the second eluate and the third eluate.

43. An aqueous IgG solution comprising at least about 99.5% IgG and less than about 0.5% impurities whereby the aqueous IgG solution comprises Factor XI as an impurity at an activity of less than about 0.0025 E/mg of total IgG, preferably less than about 0.0020 E/mg of total IgG, more preferably less than about 0.0015 E/mg of total IgG, and most preferably less than about 0.0012 E/mg of total IgG.

44. An aqueous IgG solution comprising at least about 99.5% IgG and less than about 0.5% impurities whereby the aqueous IgG solution comprises Complement C3 as an impurity at a level of less than about 3.5 μg/mg of total IgG, preferably less than about 3.0 μg/mg of total IgG, more preferably less than about 2.5 μg/mg of total IgG, and most preferably less than about 2.2 μg/mg of total IgG.
